(19) United States
(12) Patent Application Publication
(10) Pub. No.: US 2026/0318920 A1
(43) Pub. Date: Oct. 15, 2026

(54) DETECTION OF LOW-ABUNDANCE PROTEINS IN BIOLOGICAL SAMPLES USING SIZE-EXCLUSION CHROMATOGRAPHY AND MASS SPECTROMETRY

(71) Applicants: Wisconsin Alumni Research Foundation, Madison, WI (US); University of Kansas, Lawrence, KS (US)

(72) Inventors: Manish Patankar, Fitchburg, WI (US); Mark Etzel, Madison, WI (US); Naviya Schuster-Little, Madison, WI (US); Rebecca Whelan, Lawrence, KS (US)

(73) Assignees: Wisconsin Alumni Research Foundation, Madison, WI (US); University of Kansas, Lawrence, KS (US)

(21) Appl. No.: 19/647,534

(22) Filed: Apr. 14, 2026

Related U.S. Application Data
(60) Provisional application No. 63/789,007, filed on Apr. 15, 2025.

(51) Int. Cl. G01N 33/68; G01N 1/34

(52) U.S. Cl. CPC G01N 33/6848; G01N 1/34; G01N 2333/4725

(57) ABSTRACT

Methods of detecting and/or quantitating low-abundance proteins in biological samples using size-exclusion chromatography and mass spectrometry. The methods can include subjecting a biological sample comprising a target protein to size-exclusion chromatography, obtaining from the size-exclusion chromatography a size-exclusion chromatography sample comprising the target protein, preparing from the size-exclusion chromatography sample a mass-spectrometry sample comprising the target protein or a fragment thereof, and detecting the target protein or the fragment thereof in the mass-spectrometry sample using mass spectrometry. The target protein can comprise MUC16, among other proteins, and the biological sample can comprise a blood sample, such as plasma or serum.

### CROSS-REFERENCE TO RELATED APPLICATIONS

Priority is hereby claimed to U.S. Provisional Application 63/789,007, filed Apr. 15, 2025, which is incorporated herein by reference in its entirety.

### STATEMENT REGARDING FEDERALLY SPONSORED RESEARCH

This invention was made with government support under CA267532 and GM152280 awarded by the National Institutes of Health. The government has certain rights in the invention.

### FIELD OF THE INVENTION

The invention is directed to the detection and, optionally, quantitation of low-abundance proteins such as MUC16 in biological samples such as blood samples using size-exclusion chromatography and mass spectrometry.

### BACKGROUND

A persistent challenge in clinical chemistry is the detection of trace proteins in serum via mass spectrometry. The proteins present in serum span a concentration range of 10 orders of magnitude, and direct detection across this vast range challenges the dynamic response attainable by mass spectrometers.¹⁻³ Ten highly abundant proteins—albumin, IgG, IgA, IgM, transferrin, fibrinogen, alpha-2-macroglobulin, alpha-1-antitrypsin, C3 complement, and haptoglobin—account for 90% of the total protein mass in serum.⁴ Of the remaining 10%, 12 proteins make up 90% of the total.⁴ The remaining 1% of proteins found in serum appear at high dilution but are clinically and analytically significant as this protein cohort contains established and emerging biomarkers of disease. Trace serum proteins are currently used as biomarkers for prostate, liver, ovary, breast, pancreas, and colon cancer.⁵ Efforts are underway to identify new biomarkers to complement established assays.⁶⁻⁸

One long-established serum biomarker is CA125, which is approved by the FDA as an ovarian cancer marker to monitor patient response to treatment and to surveil patients in remission for disease recurrence.⁹⁻¹³ CA125 is a peptide epitope located on the mucin MUC16,^{12, 14, 15} a large, heavily glycosylated transmembrane protein composed of more than 15,000 amino acids.¹⁶ MUC16 contains three domains: an N-terminal domain of ~12,100 amino acids, a tandem-repeat region containing 19 similar, but not identical, subdomains, and a C-terminal domain that spans the cell membrane (FIG. 1). The CA125 epitope has been determined to be located within the tandem repeat domain, but the precise amino acid composition of the CA125 epitope(s) remains an area of active investigation.^{17, 18} Previous work used western blotting, ELISA, and SPR spectroscopy studies on recombinantly expressed tandem repeat proteins to demonstrate that the CA125 epitope is not uniformly distributed within the tandem repeat domain, and that the antibodies used in the clinical immunoassay—M11 and OC125—do not uniformly recognize tandem repeat domains of MUC16.^{17, 19} Glycan modification, alternative splicing of mRNA, and proteolytic cleavage may generate many MUC16 proteoforms¹³ of which only a subset are detected in the CA125 immunoassay, which may prevent early detection of ovarian cancer recurrence. A mass spectrometry-based assay for MUC16 would have great clinical value if it enabled detection of regions of the protein that are analytically silent in the existing immunoassay.

MUC16 is found in serum but is not frequently detected with mass spectrometry because of its high dilution. To enable detection of MUC16 from serum using mass spectrometry, a method is needed to enrich MUC16 from volumes of serum obtained from a clinical blood draw. Such enriched samples would contain a simpler serum proteome from which proteins of small size and high abundance have been removed while retaining MUC16 as a large and trace protein target of interest.

### SUMMARY OF THE INVENTION

One aspect of the invention is directed to detecting and/or quantitating a target protein comprised by a biological sample.

In some versions, the methods comprise subjecting a biological sample comprising a target protein to size-exclusion chromatography, obtaining from the size-exclusion chromatography a size-exclusion chromatography sample comprising the target protein, preparing from the size-exclusion chromatography sample a mass-spectrometry sample comprising the target protein or a fragment thereof, and detecting the target protein or the fragment thereof in the mass-spectrometry sample using mass spectrometry. In some versions, the methods further comprise quantitating the target protein or the fragment thereof in the mass-spectrometry sample using mass spectrometry.

In some versions, the size-exclusion chromatography sample comprises a void-peak sample.

In some versions, the size-exclusion chromatography is employed with a resin having a globular protein permeation limit less than 250 kDa. In some versions, the size-exclusion chromatography is employed with a resin having a globular protein exclusion limit of at least 2,500 kDa. In some versions, the size-exclusion chromatography is employed with a resin having a globular protein exclusion limit up to 40,000 kDa. In some versions, the size-exclusion chromatography is performed with an elution buffer having a molar ionic strength from 0.13 to 0.17 M.

In some versions, the biological sample is not subjected to ion exchange chromatography prior to the size-exclusion chromatography. In some versions, the biological sample is not subjected to protein adsorption depletion prior to the size-exclusion chromatography.

In some versions, the size-exclusion chromatography sample, the mass-spectrometry sample, and any processed forms thereof are not subjected to protein adsorption depletion prior to the detecting.

In some versions, the preparing the mass-spectrometry sample comprises subjecting the size-exclusion chromatography sample to protein adsorption depletion, wherein the protein adsorption depletion specifically targets any 1 or more, any 2 or more, any 3 or more, any 4 or more, any 5 or more, any 6 or more, any 7 or more, any 8 or more, any 9 or more, any 10 or more, any 11 or more, any 12 or more, any 13 or more, or each of albumin, IgA, IgD, IgE, IgG, IgG free light chains, IgM, alpha-1-acid glycoprotein, alpha-1-antitrypsin, alpha-2-macroglobulin, apolipoprotein A1, fibrinogen, haptoglobin, and transferrin.

In some versions, the biological sample is a direct biological fluid sample or an indirect biological sample prepared from a direct biological fluid sample. In some versions, the biological sample is selected from a blood sample and an ascites fluid sample. In some versions, the biological sample is a blood sample. In some versions, the blood sample is plasma or serum. In some versions, the biological sample is an ascites fluid sample. In some versions, an amount of the biological sample subjected to the size-exclusion chromatography corresponds to a volume of a direct biological fluid sample no greater than 5 mL.

In some versions, the target protein is MUC16 and/or any one or more proteins selected from Protein Lists 1-7.

In some versions, the target protein is a mucin.

In some versions, the target protein is MUC16. In some versions, the size-exclusion chromatography sample has CA125 purity at least 20-fold higher than CA125 purity of the biological sample. In some versions, the method is capable of enriching CA125 purity in the size-exclusion chromatography sample with respect to the biological sample at CA125 concentration ranges in the biological sample ranging from 30 U/mL to 30,000 U/mL. In some versions, the method is capable of detecting MUC16 at CA125 concentration ranges in the biological sample ranging from 30 U/mL to 30,000 U/mL. In some versions, the method further comprises quantitating the MUC16 in the mass-spectrometry sample using mass spectrometry, wherein the quantitation of MUC16 with the mass spectrometry correlates with immunoassay CA125 counts in the biological sample at CA125 concentration ranges in the biological sample ranging from 30 U/mL to 30,000 U/mL.

In some versions, the size-exclusion chromatography is performed with a disposable size-exclusion chromatography column. In some versions, the size-exclusion chromatography is performed with a single-use capable size-exclusion chromatography column. In some versions, the size-exclusion chromatography is performed with a size-exclusion chromatography column fed by gravity flow. In some versions, the size-exclusion chromatography is performed with a size-exclusion chromatography column having a length less than 80 cm.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Schematic diagram showing the domains of MUC16, the CA125 epitopes, and the clinically used antibodies

FIG. 2. Workflow and exemplary chromatography. (A) The workflow used to enrich MUC16 from biofluids. (B) A representative size-exclusion chromatogram showing absorbance at 280 nm as a function of fraction number. Fractions corresponding to the first peak were pooled and either analyzed with bottom-up proteomics directly or further processed using a Top 14 depletion column before proteomics analysis.

FIG. 3. Normalized size-exclusion chromatograms collected for four loaded volumes of plasma-derived serum. Absorbance at 280 nm was measured for each fraction. The inset shows the unretained void peak sample that contains MUC16.

FIG. 4. Sequence coverage maps of MUC16 peptides identified from plasma-derived serum. Green vertical bars indicate MUC16 peptides identified in mass spectrometry analysis.

FIG. 5. Venn diagrams comparing MUC16 peptides identified in four plasma-derived serum samples. Samples were spiked with CA125 (13, 21, 119, and 1040 U, respectively), enriched through SEC, and analyzed via bottom-up proteomics. The number of MUC16 peptides identified correlate with CA125 counts, and the peptides identified in samples with lower CA125 counts are a subset of those in samples with higher CA125 counts.

FIGS. 6A-6D. Number of peptides (log scale) identified versus the molecular weight of the parent protein for plasma-derived serum samples containing decreasing amounts of CA125: 1,040 U (FIG. 6A), 119 U (FIG. 6B), 21 U (FIG. 6C), and 13 U (FIG. 6D). The inset shows proteins of molecular weight 9-100 kDa. Red diamonds represent proteins identified in biological replicate one and blue diamonds are proteins identified in biological replicate two.

FIG. 7. Venn diagrams comparing MUC16 peptides detected through bottom-up proteomics analysis of samples enriched through SEC alone and through SEC and Top-14 protein depletion. Peptides detected in samples enriched through SEC are labeled as SEC Only (red regions of the diagram). Peptides detected in samples enriched through SEC and Top-14 depletion are labeled as SEC+DEP Only (blue regions of the diagram). Peptides common to both samples are labeled as Shared (purple regions of the diagram). Samples were prepared with CA125 concentrations ranging from 30 U/mL to 30,000 U/mL. The results of two biological replicates are shown.

FIGS. 8A and 8B. Number of MUC16 peptides identified using mass spectrometry versus amount of CA125 digested. FIG. 8A. MUC16 peptides identified after SEC only. FIG. 8B. MUC16 peptides identified after SEC and Top 14 abundant protein depletion. Each data point represents an average of technical triplicate mass spectrometry runs. Error bars are standard deviations (n=3). Slopes of two biological duplicate plots agree as determined by T-score (Panel A, SEC only: T-calc=0.0362, T-crit=2.776; p value=0.973. Panel B, SEC+Depletion: T-calc=0.073, T-crit=2.776; p value=0.946).

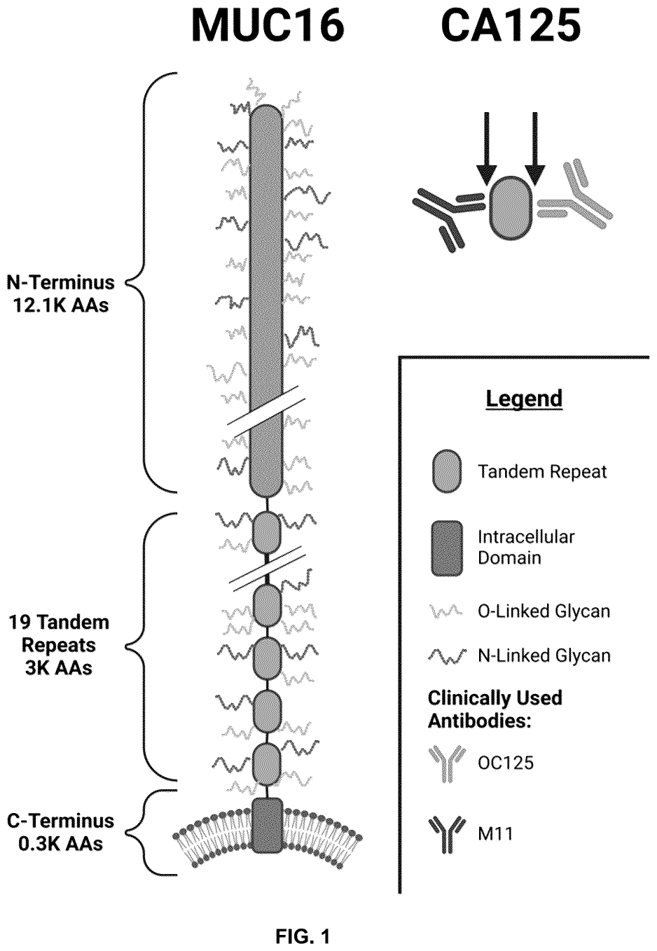

### DETAILED DESCRIPTION OF THE INVENTION

One aspect of the invention is directed to methods of detecting and/or quantitating a target protein comprised by a biological sample.

The methods can comprise a step of subjecting a biological sample comprising a target protein to size-exclusion chromatography.

“Biological sample” as used herein refers to a sample directly obtained from or derived from a biological organism, such as a plant, an animal, a mammal, or a human. The biological sample can undergo one or more processing steps between obtaining it from the biological organism and subjecting it to the size-exclusion chromatography. The processing steps can include filtration, centrifugation, addition of buffers, addition of salts, removal of components, addition of components, and coagulation, among other steps. The biological sample can accordingly be a direct biological sample or an indirect biological sample. Direct biological samples are unprocessed forms of samples taken directly from an organism. Examples include whole blood, lymph, cerebrospinal fluid, saliva, tears, synovial fluid, urine, mucus, interstitial fluid, cerebrospinal fluid, synovial fluid, pleural fluid, milk, amniotic fluid, and ascites fluid, among others. Indirect biological samples are processed forms of direct biological samples. Examples include plasma and serum.

In some versions, the biological sample is a biological fluid sample. Biological fluid samples are samples that are, comprise, or are derived from a bodily fluid. Exemplary biological fluid samples include whole blood, plasma, serum, lymph, cerebrospinal fluid, saliva, tears, synovial fluid, urine, mucus, interstitial fluid, cerebrospinal fluid, synovial fluid, pleural fluid, milk, amniotic fluid, and ascites fluid, among others. The biological fluid sample can be a direct biological fluid sample or an indirect biological fluid sample. Direct biological fluid samples are unprocessed forms of bodily fluids taken directly from an organism. Examples include whole blood, lymph, cerebrospinal fluid, saliva, tears, synovial fluid, urine, mucus, interstitial fluid, cerebrospinal fluid, synovial fluid, pleural fluid, milk, amniotic fluid, and ascites fluid, among others. Indirect biological fluid samples are processed forms of direct biological fluids. Examples include processed forms of whole blood, such as serum and plasma and processed forms of ascites fluid, such as filtered and/or centrifuged ascites fluid. In some versions, the biological fluid sample comprises a blood sample. “Blood sample” refers to whole blood or any processed form derived therefrom, including plasma and serum. In some versions, the biological fluid sample comprises an ascites sample. “Ascites sample” refers to ascites as directedly isolated from an organism or any processed form derived therefrom, such as filtered or centrifuged ascites fluid.

In some versions of the invention, the amount of the biological sample subjected to the size-exclusion chromatography corresponds to a particular volume of a direct biological fluid. “Corresponds to” in this context refers to an amount of the biological sample that is or is generated from a given amount of a direct biological fluid sample. In the degenerate case in which the biological sample is itself a direct biological fluid, the corresponding amount of the biological sample is simply the amount of the direct biological fluid sample. In a case in which the biological sample is an indirect biological sample, the corresponding amount of the indirect biological sample is a given amount of the indirect biological sample that is generated from a given amount of the direct biological fluid sample. For example, if 4.8 mL of total serum is generated from 5.0 mL of whole blood, 4.8 of the serum corresponds to 5.0 mL of the whole blood, 2.4 mL of the serum corresponds to 2.5 mL of the whole blood, and 1.2 mL of the serum corresponds to 1.25 mL of the whole blood, etc.

Some versions of the invention are particularly suited to detecting target proteins, such as low-abundance target proteins, in biological samples prepared from small-volume direct biological fluid samples. Accordingly, in various versions of the invention, the amount of the biological sample subjected to the size-exclusion chromatography corresponds to a volume of a direct biological fluid sample no greater than 1,000 mL, no greater than 900 mL, no greater than 800 mL, no greater than 700 mL, no greater than 600 mL, no greater than 500 mL, no greater than 400 mL, no greater than 300 mL, no greater than 200 mL, no greater than 100 mL, no greater than 95 mL, no greater than 90 mL, no greater than 85 mL, no greater than 80 mL, no greater than 75 mL, no greater than 70 mL, no greater than 65 mL, no greater than 60 mL, no greater than 55 mL, no greater than 50 mL, no greater than 45 mL, no greater than 40 mL, no greater than 35 mL, no greater than 30 mL, no greater than 25 mL, no greater than 20 mL, no greater than 15 mL, no greater than 10 mL, no greater than 9 mL, no greater than 8 mL, no greater than 7 mL, no greater than 6 mL, no greater than 5 mL, no greater than 4 mL, no greater than 3 mL, no greater than 2 mL, no greater than 1 mL, no greater than 0.9 mL, no greater than 0.8 mL, no greater than 0.7 mL, no greater than 0.6 mL, no greater than 0.5 mL, no greater than 0.4 mL, no greater than 0.3 mL, no greater than 0.2 mL, or no greater than 0.1 mL. In various versions of the invention, the amount of the biological fluid sample subjected to the size-exclusion chromatography corresponds to a volume of a direct biological fluid sample of at least 0.01 mL, at least 0.02 mL, at least 0.03 mL, at least 0.04 mL, at least 0.05, at least 0.06 mL, at least 0.07 mL, at least 0.08 mL, at least 0.09 mL, or at least 0.1 mL.

As is known in the art, size-exclusion chromatography is a method of separating molecules based on size and/or shape by flowing them in a liquid mobile phase (sometimes referred to as an elution buffer) through beads of resins having a range of pore sizes. The resins employed in size-exclusion chromatography typically have a fractionation range. The fractionation range indicates the sizes of molecules that can be separated by the range of pore sizes in the resin. The fractionation range can be indicated by a molecular weight range of globular proteins, molecular weight range of dextrans, or by other metrics. The lower limit of the fractionation range is referred to as the permeation limit. Molecules below the permeation limit are small enough to penetrate all the pores of the resin and typically elute in a single band at the end of elution. The upper limit of the fractionation range is referred to as the exclusion limit. Molecules above the exclusion limit have no or limited access to the resin pores and typically elute together in or just after the void volume of the column.

In various versions of the invention, the resin used in the size-exclusion chromatography has a globular protein permeation limit less than 2,250 kDa, less than 2,000 kDa, less than 1,750 kDa, less than 1,500 kDa, less than 1,250 kDa, less than 1,000 kDa, less than 950 kDa, less than 900 kDa, less than 850 kDa, less than 800 kDa, less than 750 kDa, less than 700 kDa, less than 650 kDa, less than 600 kDa, less than 550 kDa, less than 500 kDa, less than 450 kDa, less than 400 kDa, less than 350 kDa, less than 300 kDa, less than 250 kDa, less than 200 kDa, less than 150 kDa, less than 150 kDa, less than 145 kDa, less than 140 kDa, less than 135 kDa, less than 130 kDa, less than 125 kDa, less than 120 kDa, less than 115 kDa, less than 110 kDa, less than 105 kDa, less than 100 kDa, less than 95 kDa, less than 90 kDa, less than 85 kDa, less than 80 kDa, less than 75 kDa, less than 70 kDa, less than 65 kDa, less than 60 kDa, less than 55 kDa, less than 50 kDa, less than 45 kDa, less than 40 kDa, less than 35 kDa, less than 30 kDa, less than 25 kDa, less than 20 kDa, less than 15 kDa, less than 10 kDa, less than 5 kDa, or less than 1 kDa. In various versions of the invention the resin used in the size-exclusion chromatography has a globular protein permeation limit of at least 0.01 kDa, at least 0.05 kDa, at least 0.1 kDa, at least 1 kDa, at least 5 kDa, at least 10 kDa, at least 15 kDa, at least 20 kDa, at least 25 kDa, at least 30 kDa, at least 35 kDa, at least 40 kDa, at least 45 kDa, at least 50 kDa, at least 55 kDa, at least 60 kDa, at least 65 kDa, at least 70 kDa, at least 75 kDa, at least 80 kDa, at least 85 kDa, at least 90 kDa, at least 95 kDa, at least 100 kDa, at least 150 kDa, at least 200 kDa, at least 250 kDa, at least 300 kDa, at least 350 kDa, at least 400 kDa, at least 450 kDa, at least 500 kDa, at least 550 kDa, at least 600 kDa, at least 650 kDa, at least 700 kDa, at least 750 kDa, at least 800 kDa, at least 850 kDa, at least 900 kDa, at least 950 kDa, at least 1,000 kDa, at least 1,500 kDa, or at least 2,000 kDa.

In various versions of the invention, the size-exclusion chromatography is employed with a resin having a globular protein exclusion limit of at least 500 kDa, at least 750 kDa, at least 1,000 kDa, at least 1,250 kDa, at least 1,500 kDa, at least 1,750 kDa, at least 2,000 kDa, at least 2,250 kDa, at least 2,500 kDa, at least 2,750 kDa, at least 3,000 kDa, at least 3,250 kDa, at least 3,500 kDa, at least 3,750 kDa, at least 4,000 kDa, at least 4,250 kDa, at least 4,500 kDa, at least 4,750 kDa, at least 5,000 kDa, at least 5,250 kDa, at least 5,500 kDa, at least 5,750 kDa, at least 6,000 kDa, at least 6,250 kDa, at least 6,500 kDa, at least 6,750 kDa, at least 7,000 kDa, at least 7,250 kDa, at least 7,500 kDa, at least 7,750 kDa, at least 8,000 kDa, at least 8,250 kDa, at least 8,500 kDa, at least 8,750 kDa, at least 9,000 kDa, at least 9,250 kDa, at least 9,500 kDa, at least 9,750 kDa, at least 10,000 kDa, at least 15,000 kDa, at least 20,000 kDa, at least 25,000 kDa, at least 30,000 kDa, at least 35,000 kDa, at least 40,000 kDa, at least 45,000 kDa, at least 50,000 kDa, at least 55,000 kDa, at least 60,000 kDa, at least 65,000 kDa, at least 70,000 kDa, at least 75,000 kDa, at least 80,000 kDa, at least 85,000 kDa, or at least 90,000 kDa. In various versions of the invention, the size-exclusion chromatography is employed with a resin having a globular protein exclusion limit of up to 1,000 kDa, up to 1,000 kDa, up to 2,000 kDa, up to 3,000 kDa, up to 4,000 kDa, up to 5,000 kDa, up to 6,000 kDa, up to 7,000 kDa, up to 8,000 kDa, up to 9,000 kDa, up to 10,000 kDa, up to 15,000 kDa, up to 20,000 kDa, up to 25,000 kDa, up to 30,000 kDa, up to 35,000 kDa, up to 40,000 kDa, up to 45,000 kDa, up to 50,000 kDa, up to 55,000 kDa, up to 60,000 kDa, up to 65,000 kDa, up to 70,000 kDa, up to 75,000 kDa, up to 80,000 kDa, up to 85,000 kDa, up to 95,000 kDa, up to 100,000 kDa, or more.

In various versions of the invention, the exclusion limit is greater than the permeation limit by a factor of at least 2, at least 3, at least 4, at least 5, at least 6, at least 7, at least 8, at least 9, at least 10, at least 50, at least 100, at least 200, at least 300, at least 400, at least 500, at least 600, at least 700, at least 800, at least 900, at least 1,000, at least 2,500, at least 5,000, at least 7,500, or at least 10,000. In various versions of the invention, the exclusion limit is greater than the permeation limit by a factor of up to 100, up to 200, up to 300, up to 400, up to 500, up to 600, up to 700, up to 800, up to 900, up to 1,000, up to 2,500, up to 5,000, up to 7,500, up to 10,000, or up to 20,000.

In various versions of the invention, the size-exclusion chromatography is performed with an elution buffer having a molar ionic strength of at least 0.05 M, at least 0.06 M, at least 0.07 M, at least 0.08 M, at least 0.09 M, at least 0.10 M, at least 0.11 M, at least 0.11 M, at least 0.12 M, at least 0.13 M, or at least 0.14 M. In various versions of the invention, the size-exclusion chromatography is performed with an elution buffer having a molar ionic strength up to 0.16 M, up to 0.17 M, up to 0.18 M, up to 0.19 M, up to 0.20 M, up to 0.21 M, up to 0.22 M, up to 0.23 M, up to 0.24 M, or up to 0.25 M. Methods of determining ionic strength are well known in the art. See, e.g., Solomon, Theodros (2001). “The definition and unit of ionic strength.” Journal of Chemical Education. 78 (12): 1691.

The methods of the invention can comprise a step of obtaining from the size-exclusion chromatography a size-exclusion chromatography sample. “Size-exclusion chromatography sample” refers to a sample comprising one or more protein-containing eluate fractions from the size-exclusion chromatography. For use in methods of the present invention, the size-exclusion chromatography sample will comprise the target protein. In preferred versions of the invention, fractions defining the size-exclusion chromatography sample that comprise the target protein will elute prior to fractions comprising non-target proteins. Exemplary non-target proteins include albumin, IgA, IgD, IgE, IgG, IgG free light chains (e.g., kappa and lambda), IgM, alpha-1-acid glycoprotein, alpha-1-antitrypsin, alpha-2-macroglobulin, apolipoprotein A1, fibrinogen, haptoglobin, and transferrin, in any combination.

In some versions, the size-exclusion chromatography sample comprises a void-peak sample. “Void-peak sample” refers to a size-exclusion chromatography sample comprising one or more eluate fractions at least partially defining a protein peak that is at least partially defined by one or more fractions of a void volume. Thus, the void-peak sample can comprise fractions of the void volume and/or fractions eluting just after the void volume. If the latter, the protein in the fractions eluted after the void volume must define a protein peak that spans into one or more fractions of the void volume. A void volume is a portion of the elution buffer eluted from the resin prior to the elution of any protein retained by the pores of the resin. In some versions, the void-peak sample comprises at least one fraction of the void volume.

The methods of the invention can comprise a step of preparing a mass-spectrometry sample from the void-peak sample. The mass-spectrometry sample can be prepared in a manner that it comprises the target protein (e.g., for downstream “top-down” mass spectrometry analysis) or a fragment thereof (e.g., for downstream “bottom-up” mass spectrometry analysis). The preparation of the mass-spectrometry sample can comprise any of number of process steps such as buffer exchange, buffer addition, protein digestion, and ionization, among others.

In some versions, preparing the mass-spectrometry sample comprises subjecting the size-exclusion chromatography sample to protein adsorption depletion. “Protein adsorption depletion” as used herein refers to any process that binds to and removes particular proteins or types of proteins from a sample. Examples of protein adsorption depletion methods typically immobilizing a protein ligand to a solid substrate and binding the ligand to its target binding partners to remove them from a sample. Examples of protein adsorption depletion methods include immunoaffinity chromatography using antibodies and other adsorption chromatography methods employing such ligands as dye-ligands (e.g., Cibacron Blue), ion-exchange ligands, and affinity ligands such as Protein A or Protein G.

In some versions, the size-exclusion chromatography sample can be subjected to protein adsorption depletion. In some versions, the protein adsorption depletion can specifically target any 1 or more, any 2 or more, any 3 or more, any 4 or more, any 5 or more, any 6 or more, any 7 or more, any 8 or more, any 9 or more, any 10 or more, any 11 or more, any 12 or more, any 13 or more, or each of albumin, IgA, IgD, IgE, IgG, IgG free light chains (e.g., kappa and lambda), IgM, alpha-1-acid glycoprotein, alpha-1-antitrypsin, alpha-2-macroglobulin, apolipoprotein A1, fibrinogen, haptoglobin, and transferrin, in any combination. Such protein adsorption depletion can comprise immunoaffinity chromatography, among other methods. An exemplary kit for conducting such a step is the High-Select™ Top 14 Abundant Protein Depletion Resin (catalog numbers A36369, A36370, A36371, and A36372) from Pierce Biotechnology, Inc., Thermo Fisher Scientific (Rockford, Illinois, USA) Other exemplary methods and kits for conducting such a step are known in the art.

The methods of the invention can comprise a step of detecting the target protein or the fragment thereof in the mass-spectrometry sample using mass spectrometry. The detection of the target protein or fragment can comprise determining the identity or sequence of the target protein or fragment thereof. Some versions of the invention further comprise quantitating the target protein or the fragment thereof in the mass-spectrometry sample using mass spectrometry. Any type of mass spectrometry suitable for detecting and/or quantitating polypeptides in a sample can be used in the mass spectrometry step. Methods of mass spectrometry suitable for detecting and/or quantitating polypeptides in a sample are well known in the art. Exemplary methods are described elsewhere herein and are otherwise known in the art.

The target protein can comprise any one or more proteins of interest. The protein of interest can comprise an identifying feature. The identifying feature can be a structural feature, such as a particular amino acid sequence, or a functional feature, such as a particular enzymatic activity or immunoreactivity, etc. “Protein” and “polypeptide” are used interchangeably herein.

In some versions, the target protein is mucin 16 (MUC16) and/or any one or more proteins selected from Protein Lists 1-7. In some versions, the biological sample is a blood sample, and the target protein is MUC16 and/or any one or more proteins selected from Protein Lists 1-7. In some versions, the biological sample is an ascites sample, and the target protein is any one or more proteins selected from Protein Lists 2-7.

In some versions, the target protein is a mucin. The mucin can comprise any mucin. Exemplary mucins that can serve as target proteins include mucin 16 (MUC16), mucin 1 (MUC1), mucin 4 (MUC4), mucin 5B (MUC5B), and those described in Hollingsworth M A, Swanson BJ. Mucins in cancer: protection and control of the cell surface. Nat Rev Cancer. 2004 January; 4(1):45-60), among others.

In exemplary versions of the invention, the target protein is MUC16.

Versions of the invention are capable of increasing the CA125 purity of the size-exclusion chromatography sample relative to the biological sample in the size-exclusion chromatography step. CA125 purity is defined herein as units (U) of CA125 per milligram (mg) total protein in a sample. The definition of CA125 units is known in the art (Kenemans P, van Kamp G J, Oehr P, Verstraeten R A. Heterologous double-determinant immunoradiometric assay CA125 II: reliable second-generation immunoassay for determining CA125 in serum. Clin Chem. 1993 December; 39(12):2509-13). In various versions of the invention, the size-exclusion chromatography step generates size-exclusion chromatography sample having a CA125 purity at least 2-fold higher, at least 10-fold higher, at least 20-fold higher, at least 30-fold higher, at least 40-fold higher, at least 50-fold higher, at least 60-fold higher, at least 70-fold higher, at least 80-fold higher, at least 90-fold higher, at least 100-fold higher, at least 110-fold higher, at least 120-fold higher, at least 130-fold higher, at least 140-fold higher, at least 150-fold higher, at least 160-fold higher, at least 170-fold higher, at least 180-fold higher, or least 190-fold higher than the CA125 purity of the biological sample. In various versions of the invention, the size-exclusion chromatography step generates size-exclusion chromatography sample having a CA125 purity up to 150-fold higher, up to 160-fold higher, up to 170-fold higher, up to 180-fold higher, up to 190-fold higher, up to 200-fold, up to 250-fold higher or more than the CA125 purity of the biological sample. In various versions, the size-exclusion chromatography step is capable of enriching CA125 purity in the size-exclusion chromatography sample with respect to the biological sample at CA125 concentration ranges in the biological sample ranging from 30 U/mL to 30,000 U/mL.

In various versions of the invention, the methods described herein are capable of detecting MUC16 at CA125 concentration ranges in the biological sample ranging from 30 U/mL to 30,000 U/mL. “Detecting MUC16” as used herein refers to detecting the entire MUC16 protein or any fragment thereof.

Some versions of the invention comprise quantitating the MUC16 in the mass-spectrometry sample using mass spectrometry. As shown herein, the quantitation of MUC16 with the mass spectrometry correlates with immunoassay CA125 counts in the biological sample at CA125 concentration ranges in the biological sample ranging from 30 U/mL to 30,000 U/mL.

In preferred versions of the invention, the biological sample is not subjected to ion exchange chromatography prior to the size-exclusion chromatography. In some versions of the invention, the biological sample is not subjected to any protein adsorption depletion prior to the size-exclusion chromatography.

In some versions, the size-exclusion chromatography sample, the mass-spectrometry sample, and any processed forms thereof are not subjected to protein adsorption depletion prior to the detecting.

The presence of high-abundance proteins such as HSA and IgG in serum masks the detection of low-abundance proteins such as MUC16 in mass spectrometry. The top ten high-abundance proteins are 90% of the total proteins in serum and are 12 orders of magnitude more concentrated than some low-abundance proteins. The application of mass spectrometry in blood proteomics for biomarker discovery is limited by this fact of nature. Pulling MUC16 out of serum is akin to pulling a needle out of a haystack filled with high-abundance proteins. This is the reason that MUC16 is not frequently detected using mass spectrometry on serum samples. Interest in serum depletion strategies is driven by this problem. Depletion strategies have aimed at reducing the high-abundance proteins by adsorbing the high-abundance proteins from serum using immunoaffinity chromatography. Other adsorption chromatography methods have also been used such as dye-ligands (e.g. Cibacron Blue), ion-exchange ligands, and affinity ligands (e.g., Protein A or Protein G). In all these methods, the high-abundance proteins bind to an immobilized ligand and the low-abundance proteins including the target protein are washed out unbound. However, the target protein is also frequently depleted from the serum sample by non-specific binding to the bound high-abundance proteins. This co-purification results in lower yield and dilution of the target protein in the serum sample, resulting in poorer detection by mass spectrometry.

Another problem with adsorption-based depletion strategies is low capacity. For example, the maximum size immunoaffinity column available can process a maximum of 100 μL of serum. A typical blood draw produces 3-4 mL of serum, requiring 30 to 40 immunoaffinity columns where a pack of 10 columns costs $1,200. This is a direct result of the high concentration of high-abundance proteins in serum (human serum albumin alone is about 70 mg/mL in serum).

Conventional use of SEC to obtain a target protein through its fractionation leads to dilution of the target protein. Because conventional SEC fractionates the protein mixture into peaks, one of which is the target, many column volumes (CV) of buffer (about 10 CV) are typically used, which causes severe dilution of the target protein. Dilution and loss of the target protein are a problem to be minimized with all separation processes, and depletion strategies for reducing high-abundance proteins from a blood sample are no exception. Because the exemplary methods do not fractionate the target protein (MUC16), it passes through the column without significant dilution. This is an important advantage of the methods described herein.

The methods of the present invention can avoid front-end immunoaffinity chromatography, low yield, co-purification, and dilution in preparation of serum samples for detection of MUC16 and other target proteins by mass spectrometry. In some versions of the present invention, the use of size-exclusion chromatography (SEC) can serve as a one-step or first-step depletion strategy for detection of MUC16 in blood serum samples by mass spectrometry. The use of size-exclusion chromatography (SEC) as a one-step or first-step depletion strategy can achieve nearly 100% yield of MUC16, a 100-fold increase in MUC16 purity, and less than 2-fold dilution of MUC16. Optional adsorption methods can be used after the SEC step.

The ability to detect MUC16 with mass spectrometry through SEC purification without a prior adsorption step was unexpected. The present inventors expected to need an initial adsorption depletion step to clear high-abundance proteins prior to mass spectrometry. Indeed, previous attempts in purifying MUC16 from ascites employed ion-exchange chromatography followed by SEC (N. Schuster-Little, R. Fritz-Klaus, M. Etzel, N. Patankar, S. Javeri, M. S. Patankar and R. J. Whelan, Analyst, 2021, 146, 85-94). Ascites is produced in larger volumes (1.5 to 2 L), had far less high-abundance proteins than serum, and had a higher MUC16 concentration than serum. These facts made it easier to detect MUC16 in ascites, whereas detection of MUC16 in serum using mass spectrometry has been elusive in the past. The inventors first tried the ascites two-step depletion strategy (ion-exchange chromatography followed by SEC) on serum and failed. The inventors found that the bound high-abundance proteins in serum also bound MUC16, leading to co-purification. The use of SEC as a one- or first-step depletion strategy ameliorated these problems, and we successfully detected MUC16 in serum samples as small as 0.4 mL or smaller.

Also unexpected was that a short (15 cm) disposable (single-use capability) gravity-flow SEC column was sufficient to detect MUC16 in serum samples as small as 0.4 mL and as large as 2 mL. Previous work used a much longer column (100 cm) that was pump-fed and not disposable. In a clinical setting, disposable columns, syringes, and gloves are preferred to sharing or reusing medical supplies between patients. Accordingly, in some versions, the size-exclusion chromatography is performed with a disposable size-exclusion chromatography column. In some versions, the size-exclusion chromatography is performed with a single-use-capable size-exclusion chromatography column. In some versions, the size-exclusion chromatography is performed with a size-exclusion chromatography column fed by gravity flow. In some versions, the size-exclusion chromatography is performed with a size-exclusion chromatography column having a length less than 100 cm, such as less than 95 cm, less than 85 cm, less than 80 cm, less than 75 cm, less than 70 cm, less than 65 cm, less than 60 cm, less than 55 cm, less than 50 cm, less than 45 cm, less than 40 cm, less than 35 cm, less than 30 cm, less than 25 cm, less than 20 cm, or less than 15 cm. In some versions, the size-exclusion chromatography is performed with a size-exclusion chromatography column having a length of at least 1 cm, at least 2 cm, at least 3 cm, at least 4 cm, at least 5 cm, at least 6 cm, at least 7 cm, at least 8 cm, at least 9 cm, at least 10 cm, at least 11 cm, at least 12 cm, at least 13 cm, at least 14 cm, or at least 15 cm.

In some versions of the invention, the resin has a permeation limit that slows transit of these high-abundance proteins HSA (66.5 kDa) and IgG (150 kDa) through the SEC column by including these proteins in the lower end or below the fractionation range, and an exclusion limit that is orders-of-magnitude larger to speed the transit of the low-abundance proteins such as MUC16 through the SEC column. Thus, the low-abundance proteins emerge first from the SEC column, well before the emergence of the high-abundance proteins. This separation of the first peak containing the low-abundance proteins from the second peak containing the high-abundance proteins is important to increase selectivity of the separation. Selectivity is higher when there is baseline resolution between the two peaks.

In certain versions of the present methods, the target proteins (e.g., MUC16) purposefully fall at the upper end or outside the fractionation range of the resin. For the detection of MUC16, for example, the resin is chosen such that the MUC16 is too large to enter most or all of the resin pores, whereas other proteins in the mixture (such as high-abundance serum proteins) enter most or all of the resin pores and are slowed in transit through the column. Sepharose CL-4B was used in the following examples. This has a fractionation range of 60 kDa to 20,000 kDa for globular proteins such as HSA and IgG, and 30 kDa to 5,000 kDa for dextrans and unfolded proteins. The HSA (66.5 kDa) and IgG (150 kDa) fall within this fractionation range and enter the resin pores fully greatly slowing transit, whereas MUC16 (3,500 to 5,000 kDa) is heavily glycosylated and non-globular, behaving more like dextran, primarily emerging in a void volume fraction unimpeded. The procedure described herein can deplete the blood serum of the most abundant serum proteins (HSA and IgG) without fractionating them from MUC16. Other exemplary SEC resins would work to deplete the blood serum or ascites of the most abundant serum proteins (HSA and IgG) without fractionating them from MUC16. These include Sephacryl S-400 HR (globular protein fractionation range of 20 kDa to 8,000 kDa, dextran fractionation range of 10 kDa to 2,000 kDa), Superose 6 Increase (globular protein fractionation range of 5 kDa to 5,000 kDa, dextran fractionation range of 1 to 300 kDa), among others. SEC resins made of polyacrylamide, dextran-bisacrylamide, and other polymers would also be suitable.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Immunoaffinity-Free Chromatographic Purification of Ovarian Cancer Biomarker CA125 (MUC16) from Blood Serum Enables Mass Spectrometry Characterization

### Summary

The enrichment of trace proteins from human fluid samples is of great importance in diverse clinical and industrial applications. In clinical diagnostics, such enrichment may enable detection of trace proteins that serve as biomarkers of disease. Affinity-based approaches, such as immunoaffinity pulldown, are widely used to enrich trace proteins, but this strategy relies on the availability and performance of antibodies that act on all proteoforms in an unbiased manner. Our prior work to characterize mucin 16 (MUC16, the mucin protein that carries the ovarian cancer biomarker CA125) by mass spectrometry successfully overcame the reliance on affinity-based enrichment and was used to enrich this biomarker from ascites of individual ovarian cancer patients, however, this strategy was not demonstrated on clinically relevant volumes of serum, a biofluid that is more accessible than ascites. The present work developed a non-affinity-based chromatographic method to enrich MUC16 from serum. The enriched MUC16 sample was optionally further processed using a Midi Top 14 abundant protein depletion column. Peptides identified using bottom-up proteomics yielded 1-8% coverage of MUC16. Additionally, MUC16 was detected in samples containing less than the clinical cut-off level of CA125 (35 U/mL), suggesting that this strategy of enrichment and bottom-up proteomics can enable analysis of CA125 from serum of individuals with early-stage ovarian cancer and those whose tumors express CA125 (MUC16) at low levels.

### Introduction

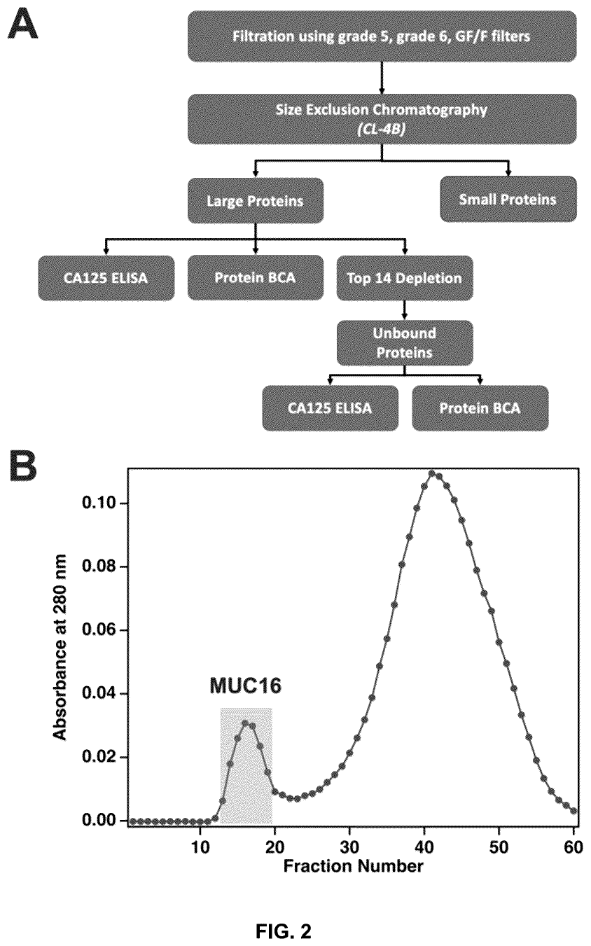

Here, we report a method that uses size-exclusion chromatography (SEC) to enrich MUC16 from biofluids including plasma-derived serum and ascites from individual patients. SEC is advantageous for enriching MUC16 which—because of its large size—can at least partially elute in a void volume that contains unretained material.^{23, 24} Samples obtained from the SEC containing MUC16 can be further enriched using Top 14 depletion columns to remove highly abundant proteins and proteins of lower abundance that are contained in larger protein complexes (FIG. 2 (A)).^{25, 26} The enrichment process reported here is compatible with 0.4-2.0 mL samples of plasma-derived serum, volumes readily obtainable with clinical blood draws, and can enrich CA125 across concentration ranges spanning clinically relevant values (30-30,000 U/mL). Using SEC followed by bottom-up proteomics, we identify peptides from MUC16 proteoforms that were enriched with and without the use of Top 14 protein depletion. We demonstrate that enrichment of MUC16 from plasma-derived serum increases analyte purity by 100-200 times over serum alone. Enriched MUC16 is detectable with mass spectrometry, and the number of MUC16 peptides detected by this method correlates with clinical CA125 counts. The method reported here is designed to enrich MUC16 proteoforms for further characterization using mass-spectrometry-based proteomics without reliance on anti-CA125-immunoaffinity enrichment that can bias the enriched sample to favor epitope-presenting proteoforms. An affinity-free enrichment and mass spectrometry approach is desirable because it enables the detection of multiple features of the MUC16 glycoprotein, in contrast with existing immunoassay methods that are limited to detecting only the CA125 epitopes. This enrichment method may enable the characterization of MUC16 from individuals with early-stage ovarian cancer and those who have tumors with low MUC16 expression.

### EXPERIMENTAL

### Chemicals and Reagents

Sodium dodecyl sulfate (SDS), iodoacetamide (IAA), triethylammonium bicarbonate (TEAB), 100 kDa molecular weight cutoff filters (MWCO), human serum, Sepharose CL-4B, and Tris base were purchased from Millipore Sigma (St. Louis, MO). Econo-Pac® Chromatography Columns were purchased from Bio-Rad (Hercules, CA). Tris (2-carboxyethyl)phosphine (TCEP), deoxycholic acid (DCA), phosphoric acid, methanol (Burdick & Jackson), and sodium chloride (NaCl) were obtained through VWR (Radnor, PA). Formic acid (99% purity; FA), acetonitrile (ACN), C18 ZipTips, Top 14 Abundant Protein Depletion Midi Spin Columns, Whatman No. 4, 5, and GF/F filter papers, Trypsin Gold, RPMI 1640 (Gibco), and L-Glutamine (Lonza) were purchased from Fisher Scientific (Hanover Park, IL). S-Traps were purchased from Protifi (Huntington, NY). The Pierce fluorometric BCA peptide assay and Pierce colorimetric BCA protein assay were purchased from Thermo Fisher (Waltham, MA). Bovine Serum Albumin was purchased from Rockland Immunochemicals Inc. (Limerick, PA). Fetal bovine serum (FBS) and 0.25% porcine trypsin were purchased from Cytiva (Marlborough, MA).

### Patient Recruitment

All experiments were approved by the Institutional Review Board of the University of Wisconsin-Madison and conducted in accordance with the U.S. Health and Human Services Basic Policy for Protection of Human Research Subjects. Ascites samples were obtained by paracentesis as a part of the standard-of-care from patients with advanced stage high-grade serous ovarian cancer who had not received any prior treatment for the cancer. The cancer diagnosis was confirmed by clinical pathology of the tumor tissue extracted through cytoreductive surgery. The ascites samples were de-identified by the UW-Madison Comprehensive Center Biobank prior to handing them over to the researchers. Within two hours of the paracentesis procedure, the ascites samples were centrifuged at 1,000×g for 20 minutes at 4° C. to remove cellular debris. The supernatants were aliquoted and stored at-80° C. until use.

Collection of MUC16 from Conditioned Cell Media

OVCAR3 cells were validated by human cell Short Tandem Repeat (STR) profiling (ATCC, Manassas, VA) and confirmed to be free of mycoplasma contamination using a PCR kit from Genlantis (San Diego, CA). Collection of MUC16 from OVCAR3 cells was performed using a method modified from Patankar and co-workers.²⁷ Briefly, cells were cultured in 175 cm² tissue culture flasks containing RPMI 1640 media supplemented with 10% FBS and 2 mM L-Glutamine. Cells were grown until they reached 100% confluency at which point spent media was removed. Cells were washed two times with RPMI 1640 media devoid of phenol red and FBS. After washing, fresh media devoid of phenol red and FBS was added. Spent media was collected after five to seven days and centrifuged for 10 minutes at 3,000×g to pellet cell debris. The supernatant was spun through a 100 kDa MWCO to concentrate MUC16 which was then spiked into plasma-derived serum as described below.

### Preparation of Ascites and Serum Samples

Prior to filtration, each ascites sample was centrifuged at 2,000×g for 30 min at 15° C. to pellet any insoluble material. Plasma-derived serum was obtained from MilliporeSigma having been prepared as follows: AB male whole blood was obtained and treated with sodium citrate to prevent coagulation. The sample was centrifuged to pellet cells and the plasma was removed. The plasma was treated with calcium to activate the coagulation cascade, centrifuged, and supernatant (serum) was removed.

Ascites and plasma-derived serum were filtered using Whatman no. 4 (25 μm), no. 5 (2.5 μm), and glass fiber (GF/F) filter (0.7 μm) papers sequentially). Filtrate from GF/F filtration was used for further sample processing. Filtered plasma-derived serum was spiked with MUC16 from the conditioned cell media of OVCAR3 cells. The volume of the spiking solution ranged from a minimum of 2% of the total volume to a maximum of 18% of the total volume. For the plasma-derived serum samples with varied CA125 counts, plasma-derived serum (diluted to 80% with RPMI 1640) was used as the diluent.

### Size-Exclusion Chromatography (SEC)

Filtered ascites or plasma-derived serum samples were loaded onto a disposable 1.5×12 cm Sepharose CL-4B gravity flow column with a fractionation range of 60 kDa to 20 MDa. The column was connected to a fraction collector (Model 2110, Bio-Rad), and fractions were collected in droplet mode. Prior to separation, the column was equilibrated using two column volumes of buffer (20 mM Tris HCl, 150 mM sodium chloride, pH 7.5). After equilibration, sample was applied to the column and allowed to enter the resin prior to separation. Proteins were eluted using equilibration buffer. Ten-droplet fractions were collected, and absorbance at 280 nm was monitored on a Nanodrop 2000 spectrometer. The fractions that made up the void peak were pooled and stored at 4° C. Total protein concentration in the pooled fractions was determined using the Pierce BCA assay. BCA assays were performed using 96-well clear plates read in a SpectraMax M5 spectrophotometer (Molecular Devices, San Jose, CA). A quadratic polynomial function was used to fit the calibration data and to calculate protein concentrations in unknown samples.

### Top 14 Abundant Protein Depletion

Three hundred microliters of slurry from a Midi Top 14 Abundant Protein Depletion Column were placed into a 10 mL Pierce centrifuge column (length=112 mm, width=16.5 mm). Three hundred micrograms of protein collected in the SEC void peak were added to this column to deplete abundant proteins. The loaded column was placed on a rotating stand at ambient temperature (approximately 22° C.) for 30 min. Following incubation, unbound proteins were eluted by centrifugation at 1,000×g for 2 min. The concentration of the eluted proteins was determined using a Pierce BCA assay.

### CA125 Quantification

CA125 counts for all samples were determined using a CA125 ELISA assay (DRG-International, Fisher Scientific) read on a SpectraMax plate reader as described above. A quadratic polynomial function was used to fit the calibration data and to calculate CA125 counts in unknown samples.

### Sample Statistics

Measured absorbance values from CA125 ELISA and protein BCA assays were used to find concentrations after fitting a quadratic polynomial to the calibration data. Uncertainties in concentrations were determined from average absorbance values using a method from the Engineering Statistics Handbook (NIST). The approach is summarized in the Description of error Propagation Methods section below. Other uncertainties were determined using the rules for error propagation.

### Description of Error Propagation Methods

The measured values from CA125 ELISA and protein BCA assays are absorbances. From these absorbance values, concentrations are inferred using a quadratic polynomial to fit the calibration data. Uncertainties in these concentrations were determined from average absorbance values using a method found in the Engineering Statistics Handbook published by the National Institutes of Standards and Technology (itl.nist.gov). Specifically, for a polynomial function of the form

Y=a+bX+cX² (Equation 1)

the quadratic formula can be written

$${X}^{′} = \frac{- b \pm \sqrt{{b}^{2} - 4 c ( a - {Y}^{′} )}}{2 a} \qquad ( Equation 2 )$$

where X′ is a calibrated value (concentration in this case) and Y′ is a measured value (absorbance in this case). The uncertainty in X′ (denoted u) is calculated from partial derivatives and the variance (u²) as follows:

$$\frac{\partial {X}^{′}}{\partial {Y}^{′}} = \frac{1}{\sqrt{{b}^{2} - 4 c ( a - {Y}^{′} )}} \qquad ( Equation 3 )$$

$$\frac{\partial {X}^{′}}{\partial {a}^{′}} = \frac{- 1}{\sqrt{{b}^{2} - 4 c ( a - {Y}^{′} )}} \qquad ( Equation 4 )$$

$$\frac{\partial {X}^{′}}{\partial b} = \frac{- 1 + \frac{b}{\sqrt{{b}^{2} - 4 c ( a - {Y}^{′} )}}}{2 c} \qquad ( Equation 5 )$$

$$\frac{\partial {X}^{′}}{\partial c} = \frac{- a + {Y}^{′}}{c \sqrt{{b}^{2} - 4 c ( a - {Y}^{′} )}} - \frac{- b + \sqrt{{b}^{2} - 4 c ( a - {Y}^{′} )}}{2 c} \qquad ( Equation 6 )$$

$${u}^{2} = {( \frac{\partial {X}^{′}}{\partial {Y}^{′}} )}^{2} {( {s}_{y} )}^{2} + {( \frac{\partial {X}^{′}}{\partial {a}^{′}} )}^{2} {( {s}_{a} )}^{2} + {( \frac{\partial {X}^{′}}{\partial {b}^{′}} )}^{2} {( {s}_{b} )}^{2} + {( \frac{\partial {X}^{′}}{\partial {c}^{′}} )}^{2} {( {s}_{c} )}^{2} \qquad ( Equation 7 )$$

$$u = \sqrt{{( \frac{\partial {X}^{′}}{\partial {Y}^{′}} )}^{2} {( {s}_{y} )}^{2} + {( \frac{\partial {X}^{′}}{\partial {a}^{′}} )}^{2} {( {s}_{a} )}^{2} + {( \frac{\partial {X}^{′}}{\partial {b}^{′}} )}^{2} {( {s}_{b} )}^{2} + {( \frac{\partial {X}^{′}}{\partial {c}^{′}} )}^{2} {( {s}_{c} )}^{2}} \qquad ( Equation 8 )$$

### Bottom-Up Proteomics Sample Preparation

Bottom-up proteomics analysis was performed on samples enriched using SEC alone and samples enriched using both SEC and Top 14 depletion. Following enrichment using SEC alone, 50 μg of total protein were digested. Following enrichment using SEC and Top 14 depletion, 30 μg of total protein were digested. Protein digestion followed methods previously described.²⁸ Briefly, proteins were denatured and reduced simultaneously using 0.4% DCA, 10% SDS, and 10 mM TCEP with incubation at 95° C. for 10 min. Afterwards, proteins were alkylated using 10 mM IAA for 30 min at ambient temperature (approximately 22° C.) in the dark. Alkylation was quenched by adding a mixture of 1.2% phosphoric acid and 90% methanol containing 100 mM TEAB to the protein solution, and the proteins were spun onto an S-Trap device. The S-Trap was washed 3 times with 90% methanol containing 100 mM TEAB. For samples that underwent SEC only, a 1:20 enzyme:protein ratio was used. Samples that underwent SEC and Top14 depletion had a 1:10 enzyme:protein ratio. For all samples, trypsin in 100 mM TEAB in water was added to the S-Trap. The digestion reaction was allowed to proceed for four hours at 37° C. Following digestion, peptides were eluted using 100 mM TEAB, 0.1% FA, and 50% ACN in three sequential steps. Eluted peptides were dried, resuspended in 0.1% FA, and desalted using ZipTips. Peptide concentrations were determined using the fluorescent peptide BCA assay. Fluorescent BCA assays were performed in 96-well black plates read in a SpectraMax M5 spectrophotometer (Molecular Devices, San Jose, CA). A quadratic polynomial function was used to fit the calibration data and to calculate peptide concentrations in unknown samples.

### Mass Spectrometry and Data Analysis

All peptide digests were analyzed using reversed-phase (RP) liquid chromatography on an UltiMate 3000 RSLCnano system coupled to an Orbitrap Eclipse Mass Spectrometer. Enriched peptide pellets were dissolved in 2% ACN/0.1% FA, and 450 ng from each sample was loaded onto an Acclaim PepMap 100 trapping cartridge using the loading pump flowing at 5 μL/min. Trapped peptides were eluted onto a 75 μm I.D.×15 cm length C18 column packed in-house with 1.9 μm ReproSil-Pur 120 C₁₈-AQ (Dr. Maisch GmbH). The mobile phases were 0.1% FA (buffer A) and 80% ACN with 0.1% FA (buffer B). Peptides were separated using a 130 min RP gradient (wash with 2% B for 5 min, 2-25% B in 82 min, 25-40% B in 20 min, 40-95% B in 1 min, hold at 95% B for 10 min, wash with 2% B for 12 min) at a flow rate of 300 nL/min. Peptides were eluted directly from the tip of the column and nano sprayed into the mass spectrometer.

The Orbitrap Eclipse was operated in data-dependent mode. Full MS¹ spectra/scans were acquired in the Orbitrap at 120K resolution (Profile mode) with mass range of 375-1500 m/z and normalized AGC target of 250%. The cycle time was set to 3 s, and within those 3 s the most abundant ions/scan were selected for fragmentation by higher energy collision dissociation (HCD). MS² spectra were collected in the ion trap in rapid scan mode (centroid mode). An isolation window of 1.6 m/z was used for the data dependent MS². Dynamic exclusion was employed for 60 s excluding all charge states for a given precursor.

RAW data files were processed using Proteome Discoverer 2.5 (PD 2.5). The data files were searched against a Homo sapiens (Hs) database and a contaminant database. The Hs database consists of 88,656 sequences, 88,654 H. sapiens non-redundant proteins (downloaded from NCBI on Apr. 11, 2022), and two additional MUC16 protein sequences. The contaminant database consists of 426 usual contaminants (human keratins, IgGs, and proteolytic enzymes). The data were searched using the SEQUEST algorithm in PD 2.5. Oxidation of methionine residues (+15.995 Da) and deamidation of asparagine, glutamine, and arginine (+0.984 Da) were set as variable modifications. Carbamidomethylation of cysteine residues (+57.021 Da) was set as a static modification. In addition, N-terminal glutamate to pyroglutamate (−17.027 Da) on peptide terminus and protein N-terminal acetylation (+42.011 Da) were set as variable modifications. Peptide-spectrum matches (PSM) were adjusted to a 1% false discovery rate (FDR) and minimum PSM number was set to two. A minimum of two peptides were required for protein identification. Data were analyzed and results generated in Excel files.

### Data Availability

The mass spectrometry proteomics data have been deposited to the ProteomeXchange Consortium via the PRIDE partner repository with the dataset identifier PXD053289.

### Results and Discussion

Enrichment of CA125 (MUC16) from Patient-Derived Ascites Using SEC

The analytical value of fractionating complex samples prior to proteomics analysis has been well demonstrated in diverse sample types including yeast,^{29, 30} Arabidopsis,^{31, 32} and ascites fluid,³³ among others. We have previously demonstrated the enrichment of MUC16 from 1.5 to 2 L of patient-derived ascites using an affinity-free method involving filtration, ion exchange chromatography (IEX), and SEC.²² The innovation of the method reported here is two-fold: (1) eliminating IEX to simplify sample processing and (2) reducing the input volumes by three orders of magnitude (to 2 mL). To determine if this method can enrich MUC16 from individual patient ascites, we processed five patient samples using SEC. Purity—defined as units (U) of CA125 per milligram (mg) total protein—for the feed solution and pooled fractions were calculated and compared. Purity was used as the metric because CA125 counts are defined in functional units and cannot be directly converted to MUC16 mass. FIG. 2 (B) shows a representative chromatogram; MUC16 is found in the first, smaller peak, which corresponds to the void volume. Table 1 reports the concentration of CA125, total protein concentration, and purity for each feed solution and resulting pooled fractions. For each pool, we also report the increase in purity achieved by SEC and the percent recovery of CA125 and total protein. SEC enrichment increases CA125 purity from a minimum of 40-fold to a maximum of 110-fold compared to the feed solution. The SEC method enriches CA125 from samples with CA125 counts spanning a wide, clinically relevant, range. Notably, the starting CA125 concentration in the ascites sample from patient 2 was less than twice the clinical cutoff (35 U/mL), suggesting that this enrichment method is compatible with the lower CA125 counts observed in healthy individuals and in early-stage ovarian cancer cases.

TABLE 1
Summary of CA125 and total protein concentrations, CA125 purity, fold enrichment from the
feed solution, and percent of CA125 and protein recovered for the individual patient ascites
samples enriched with SEC. CA125 concentrations and uncertainties were calculated from
absorbance values using the average of six reads. Protein concentrations and uncertainties
were calculated from absorbance values using the average of nine reads. Other values are
the result of calculations, with the uncertainty found from error propagation.

| | | | | Purity | | |
|---|---|---|---|---|---|---|
| | | | | Fold | | |
| Patient | CA125 | Protein | CA125 | Increase | Percent | Percent |
| Number- | Concentration | Concentration | Purity | from | Recovery | Recovery |
| Sample | (U/mL) | (mg/mL) | (U/mg) | Feed | CA125 | Protein |
| 1-Feed | 1500 ± 200 | 52 ± 2 | 30 ± 5 | | | |
| 1-Pool | 480 ± 70 | 0.283 ± 0.015 | 1700 ± 300 | 56 ± 13 | 48 ± 10 | 0.86 ± 0.06 |
| 2-Feed | 60 ± 12 | 49 ± 3 | 1.2 ± 0.3 | | | |
| 2-Pool | 20 ± 11 | 0.427 ± 0.017 | 50 ± 30 | 40 ± 20 | 60 ± 30 | 1.5 ± 0.1 |
| 3-Feed | 9000 ± 2000 | 52.3 ± 1.9 | 160 ± 30 | | | |
| 3-Pool | 3800 ± 700 | 0.304 ± 0.015 | 12000 ± 2000 | 75 ± 20 | 80 ± 20 | 1.01 ± 0.06 |
| 4-Feed | 5700 ± 1600 | 30.4 ± 1.2 | 190 ± 52 | | | |
| 4-Pool | 5000 ± 1000 | 0.258 ± 0.014 | 17000 ± 4000 | 90 ± 30 | 110 ± 40 | 1.22 ± 0.08 |
| 5-Feed | 7000 ± 2000 | 40.7 ± 1.6 | 150 ± 50 | | | |
| 5-Pool | 3400 ± 700 | 0.183 ± 0.012 | 19000 ± 4000 | 110 ± 40 | 62 ± 20 | 0.54 ± 0.04 |

Enrichment of CA125 (MUC16) from Varied Volumes of Serum

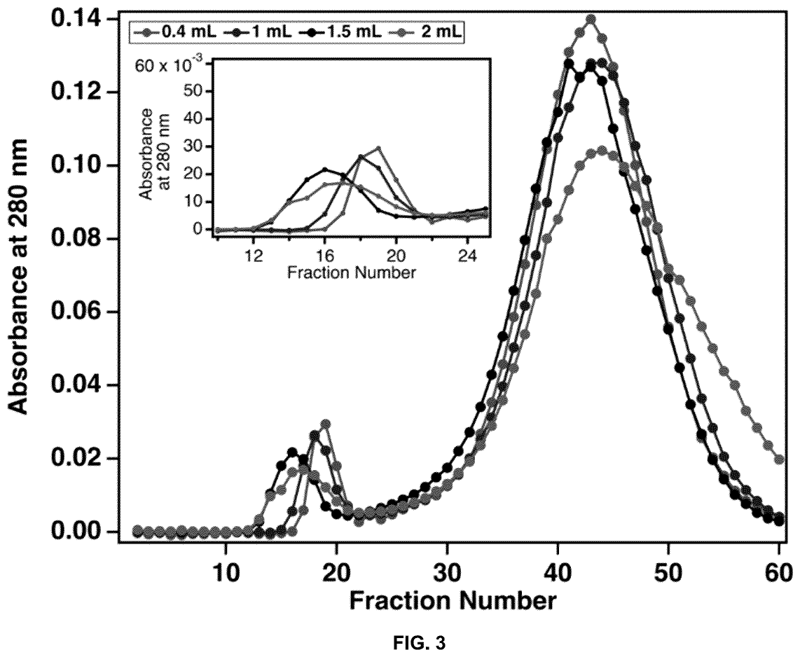

Although the SEC method reported here achieves our goal of a simple process to enrich MUC16 from ~2 mL input volumes, ascites is not observed in all ovarian cancer cases and is not found in healthy individuals. Our biofluid of ultimate interest is plasma, which can be obtained through a standard-volume blood draw (2-5 mL). We therefore sought to demonstrate the compatibility of SEC enrichment with small volumes of plasma-derived serum. To assess the appropriate amount of plasma-derived serum to load onto a 20 mL SEC column, serum samples ranging from 0.4-2.0 mL (corresponding to 2-10% column volumes) were loaded onto SEC columns in separate experiments. Prior to loading, each sample was spiked with 30,000 U/mL CA125. Resulting chromatograms displayed the general profile seen in FIG. 2 (B), with an early eluting void peak that contains MUC16 (as confirmed by CA125 ELISA), and a second, larger peak. FIG. 3 shows that when absorbance data are normalized, the chromatograms resulting from all sample volumes contain chromatographic features of comparable migration time and peak shape, suggesting that we can achieve efficient separation when 2-10% of the column volume is loaded.

Enrichment of Varied Concentrations of CA125 from Serum

Following SEC, fractions that corresponded to the void peak were pooled and designated P1, and the first five fractions of the second peak were pooled and designated P2. As for the ascites samples, purity and fold enrichment were determined. The results are reported in Table 2. For all samples, more CA125 was recovered in the void peak than the second peak, and void peak material yielded the greatest MUC16 purity. The greatest increase in purity is achieved from loading 2 mL of sample, a volume of serum attainable from a single blood draw. Serum CA125 levels can range from below 35 U/mL in healthy individuals to over 10,000 U/mL in patients with ovarian cancer. 11, 13, 34, 35 It is therefore important that an enrichment method be able to enrich MUC16 from serum samples with a wide range of CA125 concentrations. To determine the CA125 concentration range compatible with the SEC enrichment method, we prepared serum samples that contain CA125 in concentrations spanning three orders of magnitude. Samples were prepared by spiking known amounts of MUC16 into plasma-derived serum. Two mL of spiked plasma-derived serum were processed through SEC; this process was repeated a total of three times. Table 3 reports average values of concentration of CA125, total protein concentration, and purity for each feed solution and resulting pooled fractions. For each pool, we also report the increase in purity achieved by SEC and the percent recovery of CA125 and total protein. SEC enrichment increases purity between a minimum of 66-fold and a maximum of 114-fold compared to the feed solution. This purity fold enrichment compares favorably to what was observed when using SEC to enrich MUC16 from ascites.²² Additionally, most of the CA125 loaded onto the column was recovered. In contrast, less than two percent of the total protein was recovered in the void peak. This low recovery of total protein is critical to increasing the purity of MUC16.

TABLE 2
Summary of CA125 and total protein concentrations, CA125 purity, fold enrichment from the feed
solution, and percent of CA125 and protein recovered for varied volumes of plasma-derived
serum spiked with CA125. The same feed solution was used for all loading volumes. For each
volume loaded, we report data that come from the fractions corresponding to the void peak
(P1) and the following five fractions (P2). CA125 concentrations and uncertainties were calculated
from absorbance values using the average of six reads. Protein concentrations and uncertainties
were calculated from absorbance values using the average of nine reads. Other values are the
result of calculations, with the uncertainty found from error propagation.

| | CA125 | Protein | CA125 | Purity Fold | Percent | Percent |
|---|---|---|---|---|---|---|
| | Concentration | Concentration | Purity | Increase from | Recovery | Recovery |
| Sample | (U/mL) | (mg/mL) | (U/mg) | Feed | CA125 | Protein |
| 0.4 mL Feed | 17000 ± 400 | 52 ± 3 | 330 ± 20 | | | |
| 0.4 mL P1 | 1820 ± 80 | 0.038 ± 0.011 | 47000 ± 14000 | 140 ± 40 | 49 ± 3 | 0.3 ± 0.1 |
| 0.4 mL P2 | 990 ± 80 | 0.076 ± 0.011 | 13000 ± 3000 | 39 ± 7 | 32 ± 3 | 0.82 ± 0.13 |
| | | | | Total Recovery | 81 ± 11 | 1.16 ± 0.12 |
| 1.0 mL Feed | 17000 ± 400 | 52 ± 3 | 330 ± 20 | | | |
| 1.0 mL P1 | 4260 ± 170 | 0.110 ± 0.012 | 39000 ± 5000 | 118 ± 16 | 64 ± 8 | 0.05 ± 0.07 |
| 1.0 mL P2 | 2000 ± 60 | 0.233 ± 0.016 | 8600 ± 600 | 26 ± 2 | 20 ± 2 | 0.79 ± 0.07 |
| | | | | Total Recovery | 85 ± 12 | 1.33 ± 0.07 |
| 1.5 mL Feed | 17000 ± 400 | 52 ± 3 | 330 ± 20 | | | |
| 1.5 mL P1 | 5500 ± 200 | 0.0161 ± 0.013 | 34000 ± 3000 | 104 ± 11 | 70 ± 8 | 0.67 ± 0.07 |
| 1.5 mL P2 | 2700 ± 100 | 0.447 ± 0.019 | 6100 ± 300 | 19 ± 2 | 20.9 ± 1.7 | 1.12 ± 0.08 |
| | | | | Total Recovery | 91 ± 11 | 1.80 ± 0.07 |
| 2.0 mL Feed | 17000 ± 400 | 52 ± 3 | 330 ± 20 | | | |
| 2.0 mL P1 | 8200 ± 200 | 0.228 ± 0.013 | 3600 ± 2000 | 110 ± 9 | 81 ± 7 | 0.74 ± 0.06 |
| 2.0 mL P2 | 3090 ± 80 | 0.54 ± 0.03 | 5700 ± 300 | 17 ± 1 | 17.0 ± 1.4 | 0.98 ± 0.07 |
| | | | | Total Recovery | 98 ± 9 | 1.72 ± 0.06 |

TABLE 3
Summary of CA125 and total protein concentrations, CA125 purity, fold enrichment from the feed solution,
and percent of CA125 and protein recovered from samples with varied concentrations of CA125. For the
feed solutions, CA125 concentrations and uncertainties were calculated from absorbance values using
the average of six reads. Protein concentrations and uncertainties were calculated from absorbance values
using the average of nine reads. Other values are the result of calculations, with the uncertainty found
from error propagation. For each pool sample, concentrations (CA125 and protein) and associated uncertainties
were found from the calibration curve. Values in the table are the average and standard deviation for
three identical replicate samples of feed solution processed on the SEC column.

| | | CA125 | Protein | CA125 | Purity Fold | Percent | Percent |
|---|---|---|---|---|---|---|---|
| | | Concentration | Concentration | Purity | Increase | Recovery | Recovery |
| Sample | | (U/mL) | (mg/mL) | (U/mg) | from Feed | CA125 | Protein |
| 30 | U/mL Feed | 38 ± 4 | 58 ± 3 | 0.66 ± 0.07 | | | |
| 30 | U/mL Pool | 32 ± 3 | 0.248 ± 0.016 | 129 ± 16 | 196 ± 17 | 122 ± 17 | 0.62 ± 0.05 |
| 300 | U/mL Feed | 160 ± 15 | 44 ± 2 | 5.9 ± 0.4 | | | |
| 300 | U/mL Pool | 185 ± 8 | 0.253 ± 0.015 | 730 ± 50 | 123 ± 9 | 110 ± 8 | 0.89 ± 0.07 |
| 3000 | U/mL Feed | 3080 ± 180 | 45.4 ± 1.8 | 68 ± 5 | | | |
| 3000 | U/mL Pool | 1790 ± 86 | 0.267 ± 0.013 | 6700 ± 500 | 99 ± 6 | 83 ± 6 | 0.84 ± 0.05 |
| 30000 | U/mL Feed | 33000 ± 2000 | 48 ± 2 | 690 ± 50 | | | |
| 30000 | U/mL Pool | 20000 ± 1000 | 0.270 ± 0.014 | 76000 ± 5000 | 109 ± 7 | 88 ± 7 | 0.81 ± 0.06 |

SEC Enrichment of MUC16 from Serum Enables Mass Spectrometry Detection

We prepared samples of plasma-derived serum spiked with clinically relevant amounts of CA125 (5000 U/mL and 100 U/mL). These samples were analyzed via bottom-up proteomics without any prior enrichment or depletion of abundant proteins, and no MUC16 peptides were detected in either sample. These results are consistent with the findings of several recent studies performing bottom-up proteomics on serum and ascites from ovarian cancer patients that did not identify MUC16 peptides in samples with detectable CA125 counts.³⁶⁻³⁹ To determine if SEC enrichment of MUC16 enabled MUC16 peptides to be detected in a bottom-up proteomics assay, we prepared four samples spiked with varied concentrations of CA125, performed SEC enrichment as described above, and then analyzed each sample using bottom-up proteomics.

Table 4 reports CA125 and protein concentration, purity, purity fold increase over feed, and percent recoveries for these samples. The pooled fractions for each sample were proteolytically digested and analyzed using an Orbitrap Eclipse mass spectrometer. The CA125 counts in the pooled fractions ranged from a minimum of 100 U/mL to a maximum of 7,900 U/mL, which corresponds to performing digestion on CA125 in a range between a minimum of 13 U and a maximum of 1040 U.

TABLE 4
Summary of CA125 and total protein concentrations, CA125 purity, fold enrichment from
the initial sample, and percent of CA125 and protein recovered from samples with varied
concentrations of CA125 spiked into plasma-derived serum and enriched using SEC.

| | CA125 | Protein | CA125 | Purity Fold | Percent | Percent |
|---|---|---|---|---|---|---|
| | Concentration | Concentration | Purity | Increase from | Recovery | Recovery |
| Sample | (U/mL) | (mg/mL) | (U/mL) | Feed | CA125 | Protein |
| A-Feed | 26000 ± 4000 | 42.2 ± 1.9 | 520 ± 80 | | | |
| A-Pool | 7900 ± 1100 | 0.224 ± 0.013 | 35000 ± 5000 | 68 ± 18 | 56 ± 11 | 0.82 ± 0.06 |
| B-Feed | 2400 ± 300 | 41.8 ± 1.7 | 57 ± 8 | | | |
| B-Pool | 870 ± 150 | 0.208 ± 0.012 | 4200 ± 800 | 75 ± 18 | 66 ± 15 | 0.89 ± 0.06 |
| C-Feed | 620 ± 120 | 42.1 ± 1.7 | 15 ± 3 | | | |
| C-Pool | 180 ± 40 | 0.220 ± 0.014 | 810 ± 180 | 55 ± 16 | 51 ± 14 | 0.93 ± 0.07 |
| D-Feed | 200 ± 30 | 46 ± 3 | 4 ± 1 | | | |
| D-Pool | 100 ± 20 | 0.227 ± 0.016 | 500 ± 100 | 110 ± 30 | 90 ± 20 | 0.87 ± 0.08 |

Table 5 shows the CA125 concentration and amount digested along with the number of MUC16 peptides identified and percent coverage of MUC16 in duplicate digestions. MUC16 peptides were detected in all samples processed using SEC. This is in contrast with the results from samples not enriched by SEC in which no MUC16 peptides were detected in a sample that contained 5,000 U/mL CA125 (a digested amount of 22 U).

TABLE 5
Summary of mass spectrometry analysis of MUC16 enriched from
plasma-derived serum using SEC. Column 1 reports the concentration
of CA125 and Column 2 reports the amount of CA125 digested.
Columns 3 and 4 represent the average ± standard deviation
of the number of MUC16 peptides identified in three triplicate
injections (technical triplicates). Columns 5 and 6 show
the total percent coverage of MUC16.

| | CA125 | Amount | Number of | MUC16 |
|---|---|---|---|---|
| Replicate | Concentration | Digested | MUC16 | Percent |
| Number | (U/mL) | (U) | Peptides | Coverage |
| 1 | 7900 ± 1100 | 1040 | 56 ± 2 | 7 |
| 2 | | | 56 ± 3 | 8 |
| 1 | 870 ± 150 | 119 | 22 ± 2 | 5 |
| 2 | | | 29 ± 5 | 5 |
| 1 | 180 ± 40 | 21 | 3 ± 2 | 1 |
| 2 | | | 4 ± 2 | 2 |
| 1 | 100 ± 20 | 13 | 2 ± 1 | 1 |
| 2 | | | 1 ± 1 | 1 |

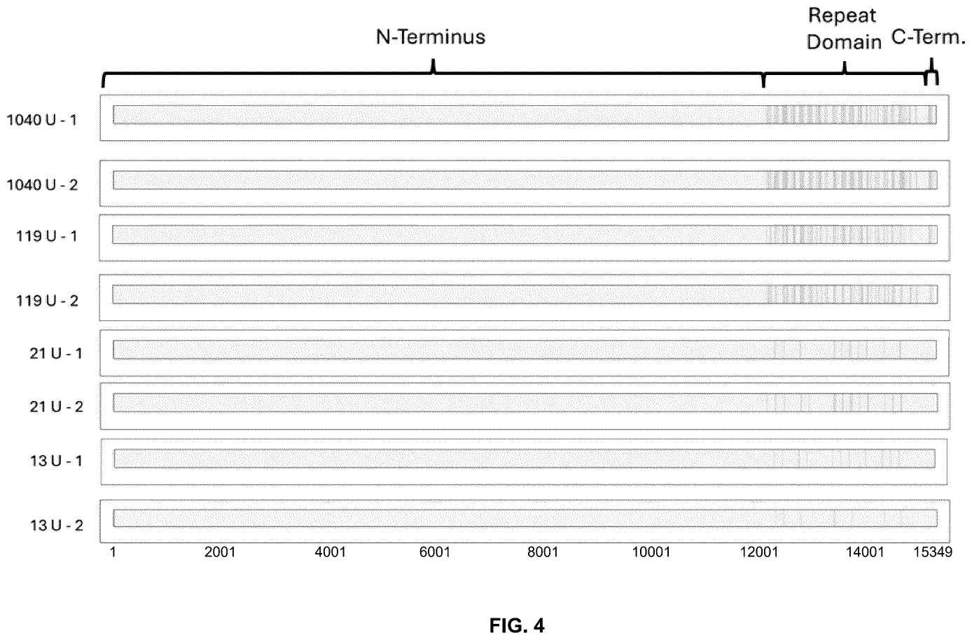

FIG. 4 shows the peptides identified in mass spectrometry (MS) mapped onto the MUC16 amino acid sequence. The number of amino acids of MUC16 reported in these coverage maps is fewer than the number used in our previous publications.^{22, 28} This change reflects our newly reported revised molecular model of the MUC16 tandem repeat domain, which contains 19 tandem repeats, not 63 as was previously reported.^{14, 16} Peptides identified through MS map to the tandem repeat domain and C-terminal domain of MUC16 and not the N-terminus, consistent with our previous reports.^{22, 28}

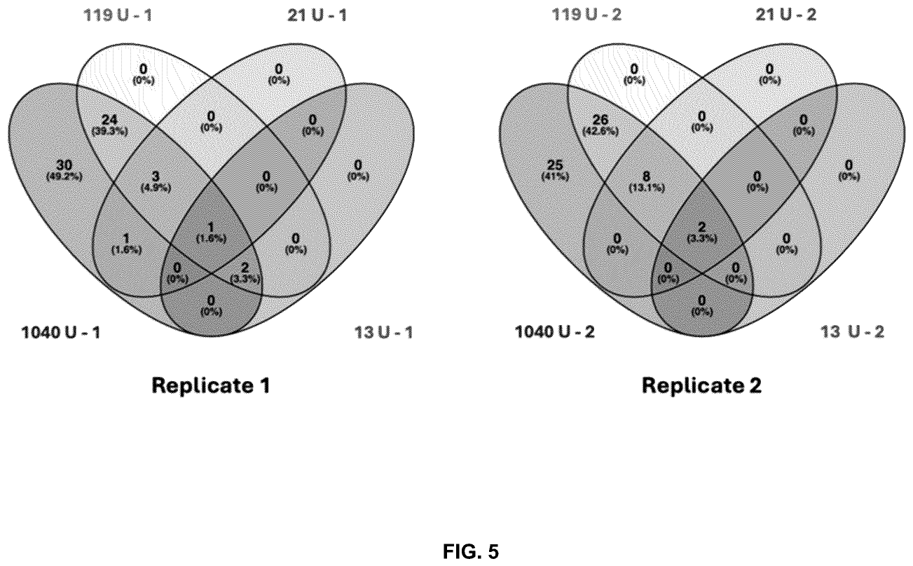

We further investigated the complementarity between peptides identified in each sample (FIG. 5). The sample that contained the highest CA125 counts resulted in the greatest number of MUC16 peptides identified. As CA125 concentration decreased, no new peptides were identified, and all identified peptides were a subset of the peptides detected in the highest concentration sample.

High Abundance, Low Molecular Weight Proteins Co-Elute with MUC16

In our previous work, low molecular weight proteins were observed to co-elute with MUC16 from a size-exclusion column. One possible explanation for this observation was that the ionic strength of the elution buffer—10 mM ammonium bicarbonate—was not sufficient to prevent protein aggregation. In this study, the input sample is serum, which has an ionic strength of ~150 mM. For the SEC separations reported here, the elution buffer—20 mM Tris HCl supplemented with 150 mM NaCl—was selected because it is isotonic with serum. When 20 mM Tris alone was used as the elution buffer, the solution was visibly cloudy, which we attribute to protein aggregation. Addition of 150 mM NaCl cleared the solution, likely because this higher ionic strength solution disrupted protein aggregation. We hypothesized that increasing the ionic strength of our buffer would also reduce the number of low molecular weight proteins that co-elute with MUC16 along with reducing protein aggregation.

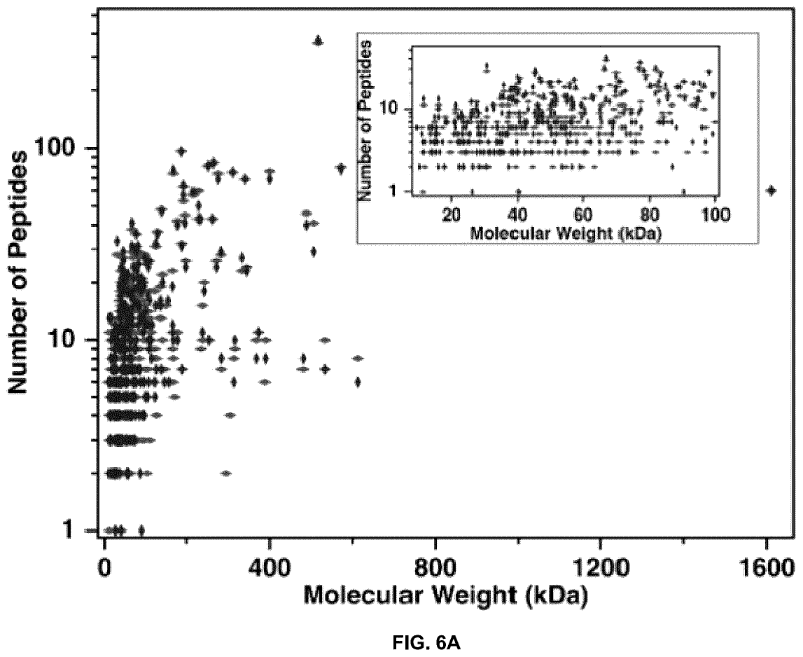

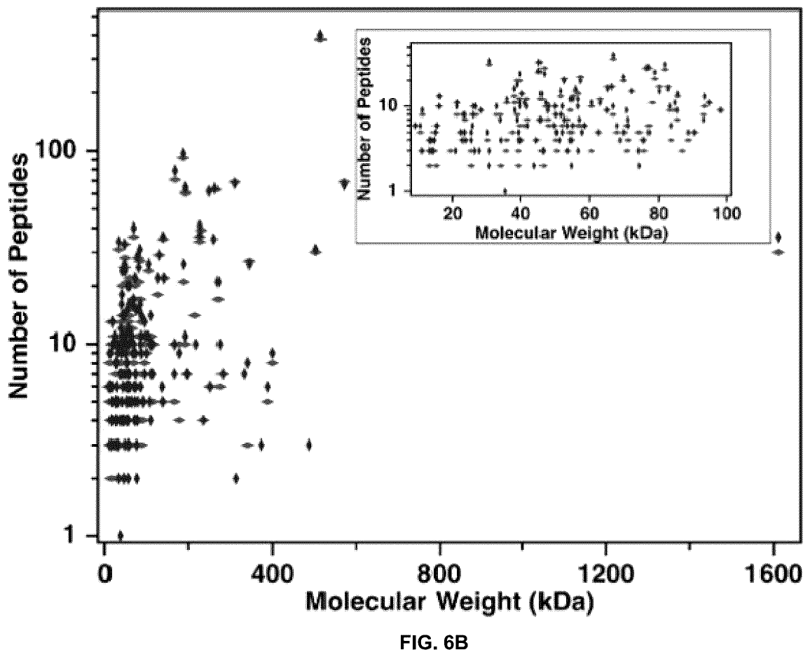

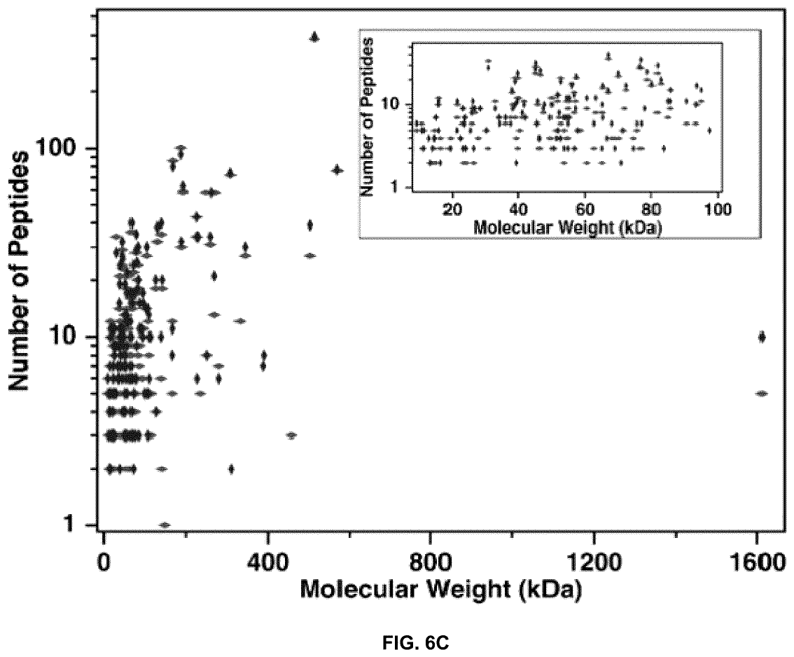

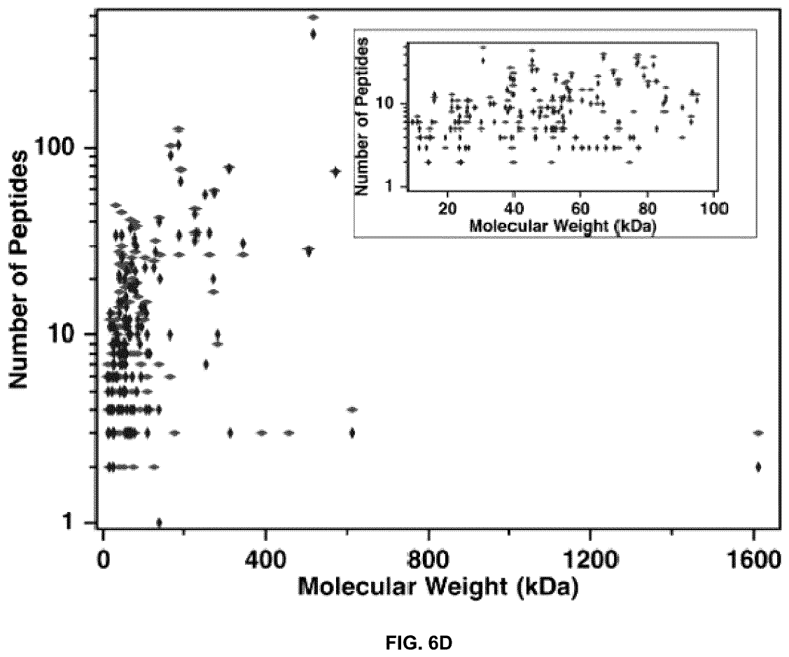

To investigate the range of molecular weights of proteins identified in mass spectrometry, the number of identified peptides originating from each protein was plotted on a log scale⁴⁰ as a function of the molecular weight of the proteins from which the peptide derived. FIGS. 6A-6D show that the molecular weight of the proteins span 9-1600 kDa. MUC16 is the only protein with a molecular weight >600 kDa. To further investigate the molecular weights of proteins identified, we examined the molecular weight of proteins ranging from 9-100 kDa (FIGS. 6A-6D, insets). Surprisingly, there is a positive correlation between the amount of CA125 digested and the number of low molecular weight proteins identified, suggesting that these proteins are co-eluting with MUC16, even at high ionic strength.

In addition to identifying low molecular weight proteins, mass spectrometry analysis shows that many highly abundant proteins are identified in the void peak. Search results identify apolipoprotein B-100, alpha-1-antitrypsin, complement factors, alpha-2-macroglobulin, and haptoglobin in all enriched samples. Although some of these proteins have a molecular weight of 400-500 kDa, several others have molecular weights <100 kDa; these proteins should have been trapped by SEC. It is possible that the proteins with molecular weights <100 kDa are co-eluting with MUC16 or forming aggregates with themselves or other proteins, including albumin.²⁵ These protein aggregates may be large enough to avoid entering the beads and elute in the void peak. We therefore explored the effects on MS of depleting abundant proteins using affinity pull-down.

### Depletion of Abundant Proteins Using Top 14 Depletion Columns

Mass spectrometry analysis reveals that high abundance proteins co-elute with MUC16 in the void peak (listed in Protein List 1). To achieve higher purity MUC16 and potentially increase the number of detectable MUC16 peptides, the SEC-enriched pool corresponding to the void peak was treated using Top 14 protein depletion columns. Surprisingly, the depletion columns were observed to deplete MUC16, perhaps via complexation of MUC16 with abundant proteins. Table 5 shows that 10-33 percent of CA125 is recovered in the flow through, significantly less than what is observed when CA125 is enriched using SEC. Additionally, Top 14 protein depletion, in contrast with SEC, does not consistently increase CA125 purity. Studies by Gundry and co-workers and Silva-Costa and co-workers have shown that commercially available abundant protein depletion columns deplete 35 to 100 more proteins than expected.^{25, 26}

Despite the decreased purity and the loss of MUC16, we hypothesized that removal of abundant proteins may enable detection of new, low-abundance MUC16 peptides that are masked by contamination with high-abundance peptides.

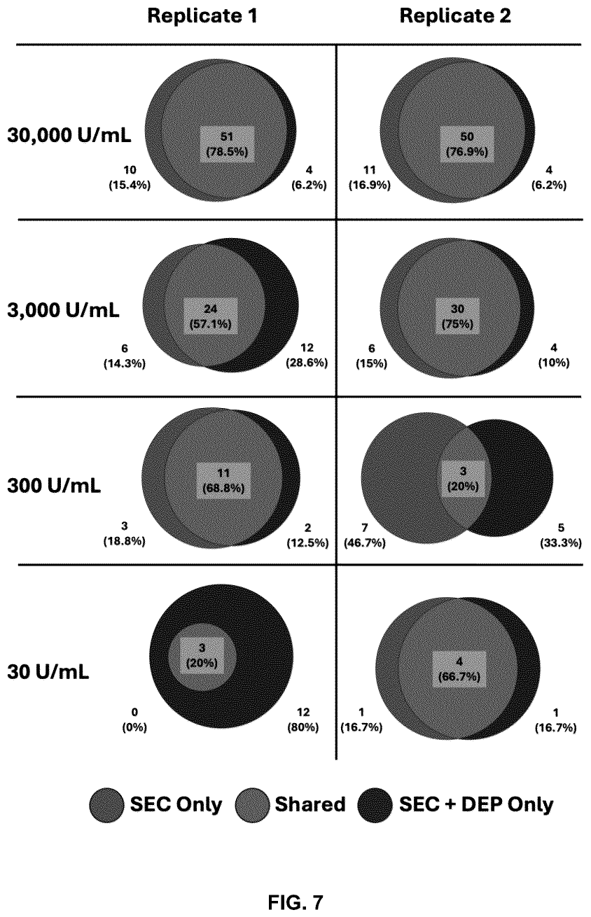

Plasma-derived serum samples that had been enriched via SEC and depletion of abundant proteins were analyzed using bottom-up proteomics. Table 6 shows a summary of the concentration and corresponding amount of CA125 digested, the number of peptides identified, and the percent coverage between two biological replicates. Table 7 shows that MUC16 peptides are identified in all samples, including those in which the CA125 concentration is below the clinical cutoff (35 U/mL). The mass spectrometry data suggest that depletion of highly abundant proteins enables detection of MUC16 peptides from lower input amounts of MUC16. A sample containing 11 U CA125 that underwent SEC and Top 14 protein depletion resulted in 4-11 identified peptides, whereas a sample that underwent SEC alone but contained a similar CA125 amount resulted in 1-2 identified peptides. The identification of a greater number of peptides when less CA125 is digested suggests that depletion of abundant proteins enables detection of more MUC16 peptides despite the penalty of MUC16 loss and may be a valuable approach, depending on the goal of the experiment. Most peptides detected through MS are shared between the two enrichment methods (FIG. 7).

TABLE 6
Summary of CA125 and total protein concentrations, CA125 purity, fold enrichment from the feed solution,
and percent of CA125 and protein recovered for samples (labelled as A-D) with varied concentrations
of CA125 that were enriched with SEC and then depleted of abundant proteins. CA125 concentrations and
uncertainties were calculated from absorbance values using the average of six reads. Protein concentrations
and uncertainties were calculated from absorbance values using the average of nine reads. Other values
are the result of calculations, with the uncertainty found from error propagation.

| | | | | Purity | | |
|---|---|---|---|---|---|---|
| | CA125 | Protein | CA125 | Fold | Percent | Percent |
| | Concentration | Concentration | Purity | Increase | Recovery | Recovery |
| Sample | (U/mL) | (mg/mL) | (U/mg) | from Feed | CA125 | Protein |
| A-SEC Only | 7900 ± 1100 | 0.224 ± 0.013 | 35000 ± 5000 | | | |
| A-SEC + Depletion | 2400 ± 100 | 0.058 ± 0.012 | 41000 ± 9000 | 1.2 ± 0.3 | 26 ± 4 | 22 ± 5 |
| B-SEC Only | 870 ± 150 | 0.208 ± 0.012 | 4200 ± 800 | | | |
| B-SEC + Depletion | 870 ± 150 | 0.049 ± 0.012 | 4000 ± 1000 | 1.0 ± 0.3 | 21 ± 4 | 21 ± 5 |
| C-SEC Only | 180 ± 40 | 0.220 ± 0.014 | 810 ± 180 | | | |
| C-SEC + Depletion | 69 ± 3 | 0.048 ± 0.017 | 1400 ± 500 | 1.8 ± 0.7 | 33 ± 7 | 19 ± 7 |
| D-SEC Only | 100 ± 20 | 0.227 ± 0.016 | 500 ± 100 | | | |
| D-SEC + Depletion | 13 ± 1 | 0.043 ± 0.011 | 300 ± 80 | 0.7 ± 0.2 | 10 ± 2 | 16 ± 4 |

TABLE 7
Summary of mass spectrometry analysis of MUC16 enriched from
plasma-derived serum using SEC followed by depletion of
Top 14 abundant proteins. Column 1 reports the concentration
of CA125 and Column 2 reports the amount of CA125 digested.
Columns 3 and 4 represent the average ± standard deviation
of the number of MUC16 peptides identified in three triplicate
injections (technical triplicates). Columns 5 and 6 show
the total percent coverage of MUC16.

| | CA125 | | Number of | |
|---|---|---|---|---|
| Replicate | Concentration | Amount | MUC16 | Percent |
| Number | (U/mL) | Digested (U) | Peptides | Coverage |
| 1 | 2400 ± 100 | 317 | 51 ± 2 | 7 |
| 2 | | | 47 ± 1 | 7 |
| 1 | 209 ± 7 | 32 | 30 ± 2 | 5 |
| 2 | | | 24 ± 2 | 5 |
| 1 | 69 ± 3 | 11 | 11 ± 1 | 2 |
| 2 | | | 4 ± 2 | 1 |
| 1 | 13 ± 1 | 2 | 11 ± 2 | 2 |
| 2 | | | 4 ± 1 | 1 |

Number of MUC16 Peptides Identified in MS Correlates with CA125 Counts

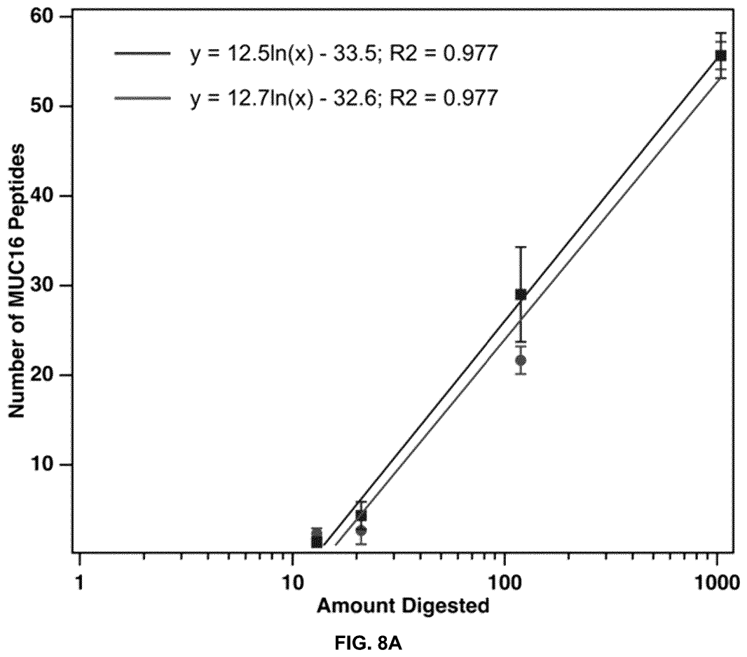

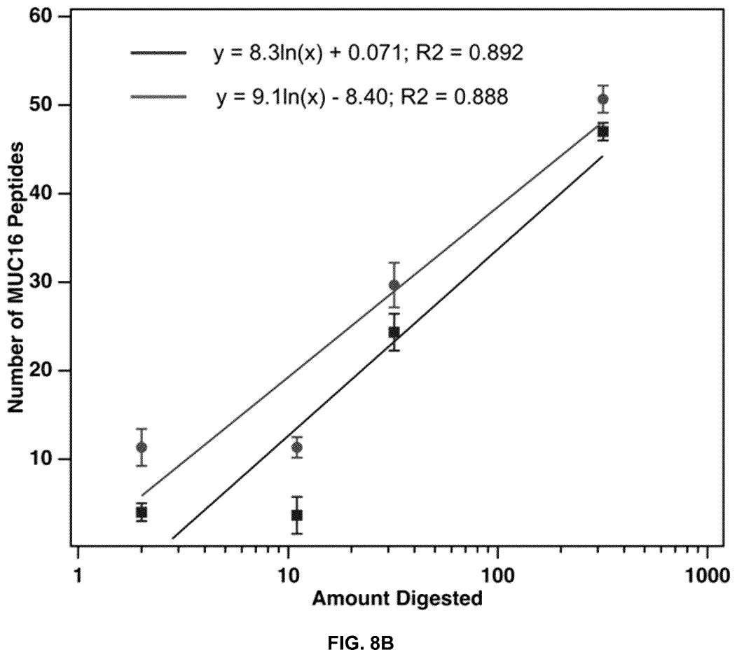

The clinical CA125 immunoassay reports concentration in units per milliliter (U/mL), and there is no conversion factor between this functional unit and amounts of MUC16 in mass or mole units. We have previously demonstrated that the number of MUC16 peptides identified correlate with clinical CA125 counts when 100-5,000 U CA125 are digested.²² To further explore the correlation of number of MUC16 peptides identified and CA125 counts, we digested 13-1040 U of CA125 enriched from plasma-derived serum using SEC. FIG. 8A shows that the number of MUC16 peptides identified from plasma-derived serum that was enriched with SEC correlates with CA125 counts determined using a research-grade CA125 ELISA. These results agree with the results of a clinical CA125 immunoassay (Table 8). FIG. 8B shows a similar trend for MUC16 peptides identified after SEC and Top 14 abundant protein depletion. This finding suggests that a mass spectrometry assay may complement the existing clinical assay by providing direct detection of MUC16 instead of the indirect detection method used in the current CA125 test. Whereas the CA125 immunoassay provides quantitative determination of the CA125 epitope(s), MS provides information on regions of MUC16 that are located both within the CA125 epitope(s) and outside. The broader molecular coverage enabled by MS therefore provides an opportunity to further stratify samples based on the biochemical presentation of MUC16.

TABLE 8
Comparison of CA125 counts obtained from a research-
grade immunoassay (reported as mean ± standard
deviation, n = 3) and a clinical immunoassay
for samples analyzed in the study.

| | | | Research | Clinical |
|---|---|---|---|---|
| | | | Grade | Test |
| | Location in | | Value | Value |
| Sample ID | Prior Table | Sample | (U/mL) | (U/mL) |
| 1F | Table 3 | 30 U/mL Feed | 38 ± 4 | 194 |
| 1P | Table 3 | 30 U/mL Pool | 32 ± 3 | 20 |
| 2F | Table 3 | 300 U/mL Feed | 160 ± 15 | 338 |
| 2P | Table 3 | 300 U/mL Pool | 185 ± 8 | 170 |
| 3F | Table 3 | 3000 U/mL Feed | 3080 ± 180 | 3132 |
| 3P | Table 3 | 3000 U/mL Pool | 1780 ± 90 | 1633 |
| 4F | Table 1 | 1 - Feed | 1500 ± 200 | 2383 |
| 4P | Table 1 | 1 - Pool | 480 ± 70 | 780 |
| 5F | Table 1 | 2 - Feed | 60 ± 12 | 71 |
| 5P | Table 1 | 2 - Pool | 20 ± 11 | 17 |
| 6F | Table 1 | 3 - Feed | 9000 ± 2000 | >10,000 |
| 6P | Table 1 | 3 - Pool | 3800 ± 700 | >10,000 |
| 7F | Table 1 | 4 - Feed | 5700 ± 1600 | >10,000 |
| 7P | Table 1 | 4 - Pool | 5000 ± 1000 | >10,000 |
| 8F | Table 1 | 5 - Feed | 7000 ± 2000 | >10,000 |
| 8P | Table 1 | 5 - Pool | 3400 ± 700 | 7885 |

### CONCLUSION

5 Using gravity flow-based size-exclusion chromatography, MUC16 can be enriched from 0.4-2.0 mL of serum and ascites. Enriched MUC16 is detectable with a high-resolution mass spectrometer, even when as little as 2 U of CA125 are digested. Although Top 14 depletion columns also remove MUC16 (an unexpected effect), more MUC16 peptides are detected in MS after Top 14 Protein depletion. The method reported here will allow for the enrichment and mass spectrometry analysis of MUC16 from serum samples of healthy patients and those with ovarian 10 cancer. This strategy enables the detection of multiple regions of MUC16, providing coverage beyond the epitope(s) recognized in the clinical CA125 test. Detection of multiple regions of MUC16 may result in a more sensitive test than the existing immunoassay and enable a new approach to stratification based on the biochemical presentation of MUC16. Additional benefit may come from replacing Data-Dependent Acquisition (DDA) with a global protein profiling approach, such as Data-Independent Acquisition (DIA), or a targeted approach, such as Parallel Reaction Monitoring (PRM). Aspects of the foregoing examples are described in Schuster-Little et al. 2024 (Schuster-Little N, Sokolovsky A D, Gentry A, Saraf A, Etzel M R, Patankar M S, Whelan R J. Immunoaffinity-free chromatographic purification of ovarian cancer biomarker CA125 (MUC16) from blood serum enables mass spectrometry characterization. Anal Methods. 2024 Sep. 26; 16(37):6337-6348), which is incorporated herein by reference.

### REFERENCES

1. L. Thadikkaran, M. A. Siegenthaler, D. Crettaz, P. A. Queloz, P. Schneider and J. D. Tissot, Proteomics, 2005, 5, 3019-3034.
2. G. Liumbruno, A. D'Alessandrd, G. Grazzini and L. Zolla, J Proteomics, 2010, 73, 483-507.
3. Y. Zhao, Q. Xue, M. Wang, B. Meng, Y. Jiang, R. Zhai, Y. Zhang, X. H. Dai and X. Fang, J Proteome Res, 2023, 22, 1009-1023.
4. R. S. Tirumalai, K. C. Chan, D. A. Prieto, H. J. Issaq, T. P. Conrads and T. D. Veenstra, Mol Cell Proteomics, 2003, 2, 1096-1103.
5. A. Tessitore, A. Gaggiano, G. Cicciarelli, D. Verzella, D. Capece, M. Fischietti, F. Zazzeroni and E. Alesse, Int J Proteomics, 2013, 2013, 125858.
6. L. Gortzak-Uzan, A. Ignatchenko, A. I. Evangelou, M. Agochiya, K. A. Brown, P. S. Onge, I. Kireeva, G. Schmitt-Ulms, T. J. Brown, J. Murphy, B. Rosen, P. Shaw, I. Jurisica and T. Kislinger, J Proteome Res, 2008, 7, 339-351.
7. S. Elschenbroich, V. Ignatchenko, B. Clarke, S. E. Kalloger, P. C. Boutros, A. O. Gramolini, P. Shaw, I. Jurisica and T. Kislinger, J Proteome Res, 2011, 10, 2286-2299.
8. C. V. Trinidad, H. B. Pathak, S. B. Cheng, S. C. Tzeng, R. Madan, M. E. Sardiu, L. E. Bantis, C. Deighan, A. Jewell, S. Rayamajhi, Y. Zeng and A. K. Godwin, Sci Rep-Uk, 2023, 13.
9. R. C. Bast, T. L. Klug, E. Stjohn, E. Jenison, J. M. Niloff, H. Lazarus, R. S. Berkowitz, T. Leavitt, C. T. Griffiths, L. Parker, V. R. Zurawski and R. C. Knapp, New Engl J Med, 1983, 309, 883-887.
10. M. E. L. Vanderburg, F. B. Lammes and J. Verweij, Ann Oncol, 1990, 1, 301-302.
11. M. E. L. Vanderburg, F. B. Lammes and J. Verweij, Neth J Med, 1992, 40, 36-51.
12. B. W. T. Yin and K. O. Lloyd, J Biol Chem, 2001, 276, 27371-27375.
13. M. Felder, A. Kapur, J. Gonzalez-Bosquet, S. Horibata, J. Heintz, R. Albrecht, L. Fass, J. Kaur, K. Hu, H. Shojaei, R. J. Whelan and M. S. Patankar, Mol Cancer, 2014, 13.
14. T. J. O'Brien, J. B. Beard, L. J. Underwood, R. A. Dennis, A. D. Santin and L. York, Tumor Biol, 2001, 22, 348-366.
15. T. J. O'Brien, J. B. Beard, L. J. Underwood and K. Shigemasa, Tumor Biol, 2002, 23, 154-169.
16. C.-W. Wang, S. D. Weaver, N. Boonpattrawong, N. Schuster-Little, M. Patankar and R. J. Whelan, Cancer Research Communications, 2024, 4, 253-263.
17. A. Bressan, F. Bozzo, C. A. Maggi and M. Binaschi, Dis Markers, 2013, 34, 257-267.
18. L. Marcos-Silva, Y. Narimatsu, A. Halim, D. Campos, Z. Yang, M. A. Tarp, P. J. B. Pereira, U. Mandel, E. P. Bennett, S. Y. Valchrushev, S. B. Levery, L. David and H. Clausen, J Proteome Res, 2014, 13, 3349-3359.
19. C. W. Wang, E. K. Hanson, L. Minkoff and R. J. Whelan, Cancer Biomark, 2023, 37, 85-94.
20. G. Becker, D. Galandi and H. E. Blum, Eur J Cancer, 2006, 42, 589-597.
21 A. A. Ayantunde and S. L. Parsons, Ann Oncol, 2007, 18, 945-949.
22. N. Schuster-Little, R. Fritz-Klaus, M. Etzel, N. Patankar, S. Javeri, M. S. Patankar and R. J. Whelan, Analyst, 2021, 146, 85-94.
23. H. M. Davis, V. R. Zurawski, Jr., R. C. Bast, Jr. and T. L. Klug, Cancer Res, 1986, 46, 6143-6148.
24. E. Perez, K. Bierla, G. Grindlay, J. Szpunar, J. Mora and R. Lobinski, Anal Chim Acta, 2018, 1018, 7-15.
25. R. L. Gundry, Q. Fu, C. A. Jelinek, J. E. Van Eyk and R. J. Cotter, Proteom Clin Appl, 2007, 1, 73-88.
26. L. C. Silva-Costa, S. Garcia-Rosa, B. J. Smith, P. A. Baldasso, J. Steiner and D. Martins-de-Souza, September Sci Plus, 2019, 2, 449-456.
27. N. K. Wong, R. L. Easton, M. Panico, M. Sutton-Smith, J. C. Morrison, F. A. Lattanzio, H. R. Morris, G. F. Clark, A. Dell and M. S. Patankar, J Biol Chem, 2003, 278, 28619-28634.
28. N. Schuster-Little, S. Madera and R. Whelan, Anal Bioanal Chem, 2020, 412, 6361-6370.
29. J. Gao, G. J. Opiteck, M. S. Friedrichs, A. R. Dongre and S. A. Hefta, J Proteome Res, 2003, 2, 643-649.
30. Q. A. Gao, A. G. Madian, X. P. Liu, J. Adamec and F. E. Regnier, J Chromatogr A, 2010, 1217, 7661-7668.
31. Z. McBride, D. L. Chen, C. Reick, J. Xie and D. B. Szymanski, Mol Cell Proteomics, 2017, 16, 1972-1989.
32. P. D. B. Olinares, L. Ponnala and K. J. van Wijk, Mol Cell Proteomics, 2010, 9, 1594-1615.
33. C. Kuk, V. Kulasingam, C. G. Gunawardana, C. R. Smith, I. Batruch and E. P. Diamandis, Mol Cell Proteomics, 2009, 8, 661-669.
34 S. J. Skates, F. J. Xu, Y. H. Yu, K. Sjovall, N. Einhorn, Y. C. Chang, R. C. Bast and R. C. Knapp, Cancer, 1995, 76, 2004-2010.
35. P. Charkhchi, C. Cybulski, J. Gronwald, F. O. Wong, S. A. Narod and M. R. Akbari, Cancers, 2020, 12.
36 J. F. Timms, E. Arslan-Low, M. Kabir, J. Worthington, S. Camuzeaux, J. Sinclair, J. Szaub, B. Afrough, V. N. Podust, E. O. Fourkala, M. Cubizolles, F. Kronenberg, E. T. Fung, A. Gentry-Maharaj, U. Menon and I. Jacobs, Proteom Clin Appl, 2014, 8, 982-993.
37. C. Han, S. Bellone, E. R. Siegel, G. Altwerger, G. Menderes, E. Bonazzoli, T. Egawa-Takata, F. Pettinella, A. Bianchi, F. Riccio, L. Zammataro, G. Yadav, J. A. Marto, M. F. Penet, D. A. Levine, R. Drapkin, A. Patel, B. Litkouhi, E. Ratner, D. A. Silasi, G. S. Huang, M. Azodi, P. E. Schwartz and A. D. Santin, Gynecol Oncol, 2018, 149, 585-591.
38. R. Bhawal, A. L. Oberg, S. Zhang and M. Kohli, Cancers, 2020, 12.
39 M. Dutt, G. Hartel, R. S. Richards, A. K. Shah, A. Mohamed, S. Apostolidou, A. Gentry-Maharaj, J. D. Hooper, L. C. Perrin, U. Menon, M. M. Hill and A. O. C. S. Grp, Proteom Clin Appl, 2023, 17.
40. T. Thavarajah, M. Tucholska, P. H. Zhu, P. Bowden and J. G. Marshall, Anal Biochem, 2020, 599, 113680.

### Protein Lists

Protein List 1 (Proteins with MW Less than 100 kDa)

14-3-3 protein beta/alpha, 14-3-3 protein epsilon, 14-3-3 protein eta, 14-3-3 protein gamma, 14-3-3 protein sigma, 14-3-3 protein theta, 14-3-3 protein zeta/delta isoform X1, 26S proteasome non-ATPase regulatory subunit 13 isoform 1, 26S proteasome non-ATPase regulatory subunit 3, 26S proteasome non-ATPase regulatory subunit 5 isoform 1, 26S proteasome regulatory subunit 7 isoform 1, 40S ribosomal protein S18, 40S ribosomal protein S2, 40S ribosomal protein S3 isoform 2, 40S ribosomal protein S3a isoform 1, 40S ribosomal protein S4, X isoform, 4F2 cell-surface antigen heavy chain isoform b, 6-phosphogluconate dehydrogenase, decarboxylating isoform 1, 60S acidic ribosomal protein P0, 60S ribosomal protein L10a, 60S ribosomal protein L5, actin-related protein 2 isoform a, actin-related protein 2 isoform b, actin-related protein 2/3 complex subunit 2, actin-related protein 3 isoform 1, actin, alpha skeletal muscle, actin, cytoplasmic 1, activator of 90 kDa heat shock protein ATPase homolog 1 isoform 1, adenosylhomocysteinase isoform X1, adipocyte plasma membrane-associated protein, adiponectin precursor, ADP-ribosylation factor 1, afamin precursor, aldehyde dehydrogenase 1A1, aldehyde dehydrogenase family 16 member A1 isoform 1, alpha-1-acid glycoprotein 1 precursor, alpha-1-acid glycoprotein 2 precursor, alpha-1-antichymotrypsin precursor, alpha-1-antitrypsin isoform X1, alpha-1B-glycoprotein precursor, alpha-2-antiplasmin isoform X2, alpha-2-HS-glycoprotein isoform 1 preproprotein, alpha-aminoadipic semialdehyde dehydrogenase isoform 1 precursor, alpha-centractin, alpha-enolase isoform 1, aminopeptidase B isoform a, angiotensinogen precursor, annexin A1 isoform X1, annexin A11 isoform X1, annexin A2 isoform 1, annexin A5, annexin A7 isoform X1, antithrombin-III isoform 3 precursor, apolipoprotein A-I isoform X1, apolipoprotein A-II preproprotein, apolipoprotein A-IV precursor, apolipoprotein A-V precursor, apolipoprotein C-I isoform a precursor, apolipoprotein C-II precursor, apolipoprotein C-III precursor, apolipoprotein C-IV precursor, apolipoprotein D precursor, apolipoprotein E isoform a precursor, apolipoprotein F preproprotein, apolipoprotein L1 isoform b precursor, apolipoprotein M isoform 1, apolipoprotein M isoform 2, argininosuccinate synthase, aspartyl aminopeptidase isoform a, ATP synthase subunit beta, mitochondrial precursor, barrier-to-autointegration factor isoform X1, basigin isoform 1 precursor, beta-2-glycoprotein 1 precursor, beta-actin-like protein 2, beta-Ala-His dipeptidase precursor, bone morphogenetic protein 7 preproprotein, BTB/POZ domain-containing protein KCTD12, C4b-binding protein alpha chain isoform X1, C4b-binding protein beta chain isoform X1, calmodulin-like protein 5, calpain-1 catalytic subunit isoform X1, carbonic anhydrase 2 isoform 1, carbonyl reductase [NADPH] 1 isoform 1, carboxypeptidase M precursor, carboxypeptidase N subunit 2 isoform X1, cartilage oligomeric matrix protein precursor, catenin alpha-1 isoform 1, catenin beta-1 isoform X1, cathelicidin antimicrobial peptide preproprotein, cathepsin D preproprotein, CD5 antigen-like isoform 1 precursor, CD5 antigen-like isoform X1, CD59 glycoprotein preproprotein, CD81 antigen isoform X1, CD82 antigen isoform X1, CD9 antigen isoform 1, cell division control protein 42 homolog isoform 1 precursor, cellular retinoic acid-binding protein 2, chloride intracellular channel protein 1, chloride intracellular channel protein 4, cholesteryl ester transfer protein isoform 1 precursor, citrate synthase, mitochondrial precursor, clathrin light chain A isoform a, claudin-3, claudin-6 precursor, clusterin preproprotein, coagulation factor IX isoform 1 preproprotein, coagulation factor XII preproprotein, cofilin-1, collagen alpha-2 (VI) chain isoform 2C₂a precursor, collectin-10 isoform 1 precursor, collectin-11 isoform f precursor, complement C1q subcomponent subunit A precursor, complement C1q subcomponent subunit B isoform 1 precursor, complement C1q subcomponent subunit C isoform 1 precursor, complement C1r subcomponent isoform 2 preproprotein, complement C1r subcomponent-like protein isoform 1 precursor, complement C1s subcomponent isoform 1 preproprotein, complement component 1 Q subcomponent-binding protein, mitochondrial precursor, complement component C7 precursor, complement component C8 alpha chain preproprotein, complement component C8 beta chain isoform 1 preproprotein, complement component C8 gamma chain precursor, complement component C9 preproprotein, complement factor B preproprotein, complement factor H-related protein 1 isoform 1 precursor, complement factor H-related protein 3 isoform 1 precursor, complement factor H-related protein 4 isoform 1 precursor, complement factor H-related protein 4 isoform X2, complement factor H-related protein 5 isoform X1, copine-1 isoform b, core histone macro-H2A.1 isoform 3, core histone macro-H2A.², cornulin, coronin-1C isoform a, cystatin-B, cystatin-SN precursor, cysteine-rich protein 2 isoform 2, D-3-phosphoglycerate dehydrogenase isoform X1, disintegrin and metalloproteinase domain-containing protein 10 isoform 1 preproprotein, DNA topoisomerase 1, DNA-(apurinic or apyrimidinic site) endonuclease, EH domain-containing protein 1 isoform 2, elongation factor 1-alpha 1, elongation factor 1-alpha 2, elongation factor 1-gamma, elongation factor 2, epithelial cell adhesion molecule precursor, eukaryotic initiation factor 4A-I isoform 1, eukaryotic initiation factor 4A-II, eukaryotic initiation factor 4A-III, eukaryotic translation initiation factor 2 subunit 3, eukaryotic translation initiation factor 2 subunit 3B isoform 1, eukaryotic translation initiation factor 3 subunit I isoform 1, eukaryotic translation initiation factor 3 subunit L isoform 1, eukaryotic translation initiation factor 6 isoform a, extracellular matrix protein 1 isoform 3 precursor, ezrin, fatty acid-binding protein 5, fermitin family homolog 3 long isoform, fibrinogen alpha chain isoform alpha-E preproprotein, fibrinogen beta chain isoform 1 preproprotein, fibrinogen gamma chain isoform gamma-B precursor, fibulin-1 isoform C precursor, fibulin-1 isoform D precursor, ficolin-2 isoform a precursor, ficolin-3 isoform 1 precursor, fructose-bisphosphate aldolase A isoform 2, fructose-bisphosphate aldolase C isoform X1, G-protein coupled receptor family C group 5 member C isoform X1, galectin-3-binding protein precursor, galectin-7, gelsolin isoform a precursor, glucose 1,6-bisphosphate synthase isoform X1, glucose-6-phosphate isomerase isoform X1, glutamate dehydrogenase 1, mitochondrial isoform a precursor, glutathione S-transferase P, glyceraldehyde-3-phosphate dehydrogenase isoform 1, glycogen phosphorylase, brain form, glycogen phosphorylase, liver form isoform 1, glypican-4 precursor, GMP synthase [glutamine-hydrolyzing] isoform X1, GTP-binding nuclear protein Ran isoform 1, guanine nucleotide-binding protein G (i) subunit alpha-2 isoform X1, guanine nucleotide-binding protein G (i) subunit alpha-3, guanine nucleotide-binding protein G (I)/G(S)/G (T) subunit beta-1 isoform X1, guanine nucleotide-binding protein G (I)/G(S)/G (T) subunit beta-2, haptoglobin isoform 1 preproprotein, haptoglobin-related protein isoform X1, heat shock 70 kDa protein 1B, heat shock 70 kDa protein 4, heat shock 70 kDa protein 6, heat shock cognate 71 kDa protein isoform X1, heat shock protein beta-1, heat shock protein HSP 90-alpha isoform 1, heat shock protein HSP 90-beta isoform a, hemoglobin subunit alpha, hemoglobin subunit beta, hemoglobin subunit delta, hemopexin precursor, heparin cofactor 2 precursor, hepatocyte growth factor activator isoform 1 preproprotein, heterogeneous nuclear ribonucleoprotein DO isoform a, heterogeneous nuclear ribonucleoprotein K isoform X1, heterogeneous nuclear ribonucleoprotein R isoform X1, heterogeneous nuclear ribonucleoprotein U isoform a, high mobility group protein B1 isoform X1, high mobility group protein B2, high mobility group protein B3 isoform X1, histidine-rich glycoprotein isoform X1, histone deacetylase 1, histone deacetylase 2, histone H1.3, histone H2A type 1-C, histone H2A type 2-A, histone H2A.Z, histone H2AX, histone H2B type 1-O, histone H2B type 2-F isoform b, histone H3.1, histone H3.2, histone H3.3, histone H4 isoform X1, HLA class I histocompatibility antigen, A alpha chain isoform X2, hyaluronan-binding protein 2 isoform 1 preproprotein, immunoglobulin heavy variable 4-38-2-like, immunoglobulin lambda-like polypeptide 5 isoform 1, immunoglobulin superfamily member 8 isoform X1, importin subunit beta-1 isoform 1, inorganic pyrophosphatase, insulin-like growth factor-binding protein complex acid labile subunit isoform 1 precursor, integrin beta-1 isoform 1A precursor, integrin beta-1 isoform 1D precursor, integrin beta-3 precursor, inter-alpha-trypsin inhibitor heavy chain H1 isoform b, isocitrate dehydrogenase [NADP] cytoplasmic, junction plakoglobin isoform X1, junction plakoglobin isoform X2, kallistatin isoform 1, kininogen-1 isoform 1 precursor, kininogen-1 isoform 2 precursor, L-lactate dehydrogenase A chain isoform 3, L-lactate dehydrogenase B chain isoform LDHBx, L-lactate dehydrogenase B chain isoform X1, lactadherin isoform a preproprotein, lactotransferrin isoform 1 preproprotein, lamin isoform A, leukocyte surface antigen CD47 isoform 1 precursor, lipopolysaccharide-binding protein precursor, lysozyme C precursor, major vault protein isoform 1, malate dehydrogenase, mitochondrial isoform 1 precursor, mannan-binding lectin serine protease 1 isoform X1, mannan-binding lectin serine protease 1 isoform X2, mannan-binding lectin serine protease 2 isoform 1 preproprotein, mannose-binding protein C precursor, mesothelin isoform 1 preproprotein, mesothelin isoform 2 preproprotein, midkine isoform a precursor, mitotic checkpoint protein BUB3 isoform a, moesin isoform X1, myeloperoxidase precursor, N-acetylmuramoyl-L-alanine amidase isoform 2 precursor, neutral amino acid transporter B (0) isoform 1, nucleolin, nucleoside diphosphate kinase A isoform a, obg-like ATPase 1 isoform 1, Parkinson disease protein 7 isoform X1, pentraxin-related protein PTX3 precursor, peptidyl-prolyl cis-trans isomerase A isoform 1, peroxiredoxin-1, peroxiredoxin-2, peroxiredoxin-6, phosphatidylcholine-sterol acyltransferase precursor, phosphatidylinositol-glycan-specific phospholipase D isoform X1, phosphoglucomutase-1 isoform 1, phosphoglycerate kinase 1, phosphoglycerate mutase 1 isoform 1, phospholipid transfer protein isoform a precursor, plasma kallikrein isoform X1, plasma kallikrein isoform X2, plasma protease C1 inhibitor precursor, plasma serine protease inhibitor preproprotein, plasminogen isoform 1 precursor, platelet factor 4 isoform 2, platelet-activating factor acetylhydrolase IB subunit beta isoform X1, platelet-activating factor acetylhydrolase isoform X1, poly (rC)-binding protein 1, polyadenylate-binding protein 1 isoform X1, prenylcysteine oxidase 1 precursor, procollagen-lysine,2-oxoglutarate 5-dioxygenase 1 isoform 1 precursor, profilin-1 isoform 2, programmed cell death 6-interacting protein isoform 1, programmed cell death 6-interacting protein isoform 2, programmed cell death protein 6 isoform 1, prolactin-inducible protein precursor, properdin precursor, prostaglandin F2 receptor negative regulator isoform X1, proteasome subunit alpha type-5 isoform 1, proteasome subunit alpha-type 8 isoform 1, protein AMBP preproprotein, protein arginine N-methyltransferase 1 isoform 1, protein disulfide-isomerase A3 precursor, protein NDRG1 isoform 1, protein NDRG1 isoform 4, protein RCC2, protein S100-A10, protein S100-A4, protein S100-A7, protein-glutamine gamma-glutamyltransferase E, prothrombin preproprotein, purine nucleoside phosphorylase, putative V-set and immunoglobulin domain-containing-like protein IGHV4OR15-8, pyruvate kinase PKM isoform X1, rab GDP dissociation inhibitor beta isoform 1, ras-related C3 botulinum toxin substrate 1 isoform Raclb, ras-related protein Rab-13 isoform 1, ras-related protein Rab-1A isoform 1, ras-related protein Rab-1B, ras-related protein Rab-7a, ras-related protein Rab-8A, ras-related protein Rap-1b isoform 1 precursor, receptor of activated protein C kinase 1, retinoic acid-induced protein 3, retinol-binding protein 4 isoform a precursor, rho-related GTP-binding protein RhoC precursor, ruvB-like 1 isoform 1, ruvB-like 2 isoform 1, S-formylglutathione hydrolase isoform X1, S-methyl-5′-thioadenosine phosphorylase isoform 6, SAA2-SAA4 protein precursor, secretory carrier-associated membrane protein 3 isoform 1, selenoprotein P isoform 2, serine hydroxymethyltransferase, mitochondrial isoform 1 precursor, serine/threonine-protein phosphatase PP1-beta catalytic subunit isoform 1, serotransferrin isoform 1 precursor, serpin H1 isoform X1, serum amyloid A-1 protein preproprotein, serum amyloid A-2 protein isoform a preproprotein, serum amyloid P-component precursor, serum paraoxonase/arylesterase 1 precursor, serum paraoxonase/lactonase 3, single-stranded DNA-binding protein, mitochondrial precursor, small nuclear ribonucleoprotein Sm D2 isoform 1, sodium-dependent phosphate transport protein 2B isoform a, sodium/potassium-transporting ATPase subunit beta-3, spliceosome RNA helicase DDX39B, sulfhydryl oxidase 1 isoform a precursor, synaptogyrin-2 isoform 3, syntenin-1 isoform 7, T-complex protein 1 subunit alpha isoform a, T-complex protein 1 subunit beta isoform 1, T-complex protein 1 subunit delta isoform a, T-complex protein 1 subunit epsilon isoform a, T-complex protein 1 subunit eta isoform a, T-complex protein 1 subunit gamma isoform a, T-complex protein 1 subunit theta isoform 1, T-complex protein 1 subunit zeta isoform a, testican-2 isoform 2 precursor, tetraspanin-14 isoform 1, threonine--tRNA ligase 1, cytoplasmic isoform 2, transcriptional activator protein Pur-alpha, transcriptional activator protein Pur-beta, transferrin receptor protein 1 isoform 1, transforming growth factor-beta-induced protein ig-h3 precursor, transforming protein RhoA isoform 1 precursor, transgelin-2 isoform a, transitional endoplasmic reticulum ATPase isoform 1, transketolase isoform X1, transthyretin precursor, triosephosphate isomerase isoform 2, tropomyosin alpha-1 chain isoform X10, tropomyosin alpha-3 chain isoform Tpm3.2cy, tubulin alpha-1A chain isoform 1, tubulin alpha-1C chain isoform a, tubulin alpha-4A chain isoform X1, tubulin beta chain isoform a, tubulin beta chain isoform b, tubulin beta-1 chain, tubulin beta-3 chain isoform 1, tubulin beta-4A chain isoform 1, tubulin beta-4B chain, tubulin beta-6 chain isoform 1, tumor-associated calcium signal transducer 2 precursor, UDP-glucose 6-dehydrogenase isoform X1, V-type proton ATPase catalytic subunit A isoform X1, vacuolar protein sorting-associated protein 35, vitamin D-binding protein isoform 3 precursor, vitamin K-dependent protein S isoform 1 precursor, vitronectin precursor, WD repeat-containing protein 1 isoform X1, X-ray repair cross-complementing protein 5, X-ray repair cross-complementing protein 6 isoform 1, Y-box-binding protein 1, zinc-alpha-2-glycoprotein precursor, and zymogen granule protein 16 homolog B precursor.

### Protein List 2 (GN=Indicates Gene Name)

Apolipoprotein B-100 GN=APOB, Complement C3 GN=C3, Complement C4-B GN=C4B, Complement C4-A GN=C4A, Fibronectin GN=FN1, Mucin-5B GN=MUC5B, Mucin-16 GN=MUC16, Alpha-2-macroglobulin GN=A2M, Ceruloplasmin GN=CP, Fibrinogen beta chain GN=FGB, Ig mu chain C region GN-IGHM, Complement C5 GN=C5, Serum albumin GN=ALB, Inter-alpha-trypsin inhibitor heavy chain H2 GN=ITIH2, Inter-alpha-trypsin inhibitor heavy chain H1 GN=ITIH1, Ig mu heavy chain disease protein, Aminopeptidase N GN=ANPEP, Plasma protease C1 inhibitor GN=SERPING1, C4b-binding protein alpha chain GN-C4BPA, Fibrinogen gamma chain GN-FGG, Complement factor H GN=CFH, Collagen alpha-3 (VI) chain GN═COL6A3, von Willebrand factor GN=VWF, Proteoglycan 4 GN-PRG4, Actin, cytoplasmic 1 GN=ACTB, Apolipoprotein A-I GN=APOA1, Apolipoprotein (a) GN-LPA, Ezrin GN=EZR, Ig alpha-1 chain C region GN=IGHA1, Pregnancy zone protein GN=PZP, Basement membrane-specific heparan sulfate proteoglycan core protein GN═HSPG2, CD5 antigen-like GN=CD5L, Complement C1r subcomponent GN-CIR, Ig kappa chain C region GN-IGKC, Sushi, von Willebrand factor type A, EGF and pentraxin domain-containing protein 1 GN=SVEP1, Fibrinogen alpha chain GN-FGA, Phospholipid transfer protein GN=PLTP, Annexin A2 GN=ANXA2, Inter-alpha-trypsin inhibitor heavy chain H4 GN=ITIH4, Complement component C9 GN=C9, Apolipoprotein E GN=APOE, Vitronectin GN=VTN, Serum paraoxonase/arylesterase 1 GN=PON1, Prothrombin GN=F2, Ig gamma-1 chain C region GN=IGHG1, Lipopolysaccharide-binding protein GN=LBP, Ig alpha-2 chain C region GN=IGHA2, Alpha-1-antitrypsin GN=SERPINA1, Versican core protein GN=VCAN, Target of Nesh-SH3 GN=ABI3BP, Clusterin GN=CLU, Haptoglobin GN=HP, Mucin-4 GN=MUC4, Inter-alpha-trypsin inhibitor heavy chain H3 GN=ITIH3, Complement C1s subcomponent GN=C1S, Heparin cofactor 2 GN=SERPIND1, Moesin GN=MSN, Ig kappa chain V-I region DEE, Annexin A1 GN=ANXA1, Vitamin K-dependent protein S GN=PROS1, Prominin-1 GN=PROM1, IgGFc-binding protein GN=FCGBP, Ig lambda-2 chain C regions GN=IGLC2, Pyruvate kinase PKM GN=PKM, Immunoglobulin lambda-like polypeptide 5 GN=IGLL5, Haptoglobin-related protein GN=HPR, Ig gamma-2 chain C region GN=IGHG2, Annexin A5 GN=ANXA5, Alpha-enolase GN=ENO1, Gelsolin GN=GSN, Collagen alpha-1 (VI) chain GN═COL6A1, Galectin-3-binding protein GN=LGALS3BP, 5′-nucleotidase GN=NT5E, Laminin subunit beta-1 GN=LAMB1, Ras GTPase-activating-like protein IQGAP1 GN=IQGAP1, Guanine nucleotide-binding protein G (i) subunit alpha-2 GN-GNAI2, Plasminogen GN=PLG, Erythrocyte band 7 integral membrane protein GN=STOM, Laminin subunit gamma-1 GN-LAMC1, Complement component C7 GN-C7, Kininogen-1 GN=KNG1, Fibulin-1 GN=FBLN1, Glyceraldehyde-3-phosphate dehydrogenase GN-GAPDH, Alpha-1-antichymotrypsin GN=SERPINA3, Alkaline phosphatase, placental type GN=ALPP, Ig kappa chain V-IV region Len, Immunoglobulin J chain GN=IGJ, Insulin-like growth factor-binding protein complex acid labile subunit GN=IGFALS, Apolipoprotein A-IV GN=APOA4, Phosphoglycerate kinase 1 GN=PGK1, Monocyte differentiation antigen CD14 GN-CD14, Ig heavy chain V-III region BUT, Ig kappa chain V-I region WEA, Myosin-9 GN=MYH9, Dipeptidyl peptidase 4 GN-DPP4, Ig kappa chain V-III region B6, Ig kappa chain V-III region GOL, Complement component C6 GN=C6, EMILIN-2 GN=EMILIN2, Actin, alpha cardiac muscle 1 GN=ACTC1, N-acetylmuramoyl-L-alanine amidase GN=PGLYRP2, Ig kappa chain V-I region OU, Ig kappa chain V-III region SIE, Serum amyloid A-1 protein GN=SAA1, Ig gamma-3 chain C region GN=IGHG3, Ig kappa chain V-I region Scw, Guanine nucleotide-binding protein G (I)/G(S)/G (T) subunit beta-2 GN=GNB2, Ig kappa chain V-I region CAR, Ig kappa chain V-I region EU, Retinoic acid-induced protein 3 GN=GPRC5A, Heat shock cognate 71 kDa protein GN═HSPA8, Ig kappa chain V-I region AU, Ig kappa chain V-I region Roy, Agrin GN=AGRN, Angiotensinogen GN=AGT, Complement component C8 beta chain GN=C8B, Histidine-rich glycoprotein GN-HRG, Ig kappa chain V-III region HIC, Guanine nucleotide-binding protein G (I)/G(S)/G (T) subunit beta-1 GN=GNB1, Apolipoprotein LI GN=APOL1, Transthyretin GN=TTR, Chloride intracellular channel protein 1 GN=CLIC1, Chloride intracellular channel protein 4 GN=CLIC4, Programmed cell death 6-interacting protein GN=PDCD6IP, Tenascin GN=TNC, Complement component C8 alpha chain GN=C8A, Complement C1q subcomponent subunit B GN-C1QB, ADP-ribosylation factor 3 GN=ARF3, Elongation factor 1-alpha 1 GN=EEF1A1, Protein AMBP GN=AMBP, Annexin A4 GN=ANXA4, 14-3-3 protein zeta/delta GN=YWHAZ, Ig kappa chain V-I region Ni, Ig kappa chain V-II region TEW, Ig heavy chain V-III region GAL, Tubulin beta chain GN=TUBB, Guanine nucleotide-binding protein G(s) subunit alpha isoforms XLas GN=GNAS, Heat shock 70 kDa protein 1A/1B GN═HSPA1A, Laminin subunit alpha-4 GN=LAMA4, Guanine nucleotide-binding protein G (q) subunit alpha GN-GNAQ, Ig heavy chain V-I region V35, 14-3-3 protein epsilon GN=YWHAE, Tubulin alpha-1B chain GN=TUBA1B, Ig heavy chain V-III region BRO, Complement C1q subcomponent subunit C GN-C1QC, Peptidyl-prolyl cis-trans isomerase A GN=PPIA, Lumican GN=LUM, Lactadherin GN=MFGE8, Alpha-2-antiplasmin GN=SERPINF2, ADP-ribosylation factor 4 GN=ARF4, Plastin-3 GN=PLS3, Ig kappa chain V-I region Wes, Brain-specific angiogenesis inhibitor 1-associated protein 2 GN=BAIAP2, Unconventional myosin-Ic GN=MYO1C, Heat shock protein HSP 90-beta GN═HSP90AB1, Ras-related protein Rap-1b GN=RAP1B, Pigment epithelium-derived factor GN=SERPINF1, Ig heavy chain V-III region CAM, EH domain-containing protein 1 GN-EHD1, L-lactate dehydrogenase A chain GN=LDHA, Ig heavy chain V-I region HG3, Carboxypeptidase N subunit 2 GN=CPN2, Epidermal growth factor receptor kinase substrate 8-like protein 1 GN=EPS8L1, Vitamin D-binding protein GN-GC, C4b-binding protein beta chain GN=C4BPB, Annexin A6 GN=ANXA6, Complement component C8 gamma chain GN=C8G, Ras-related protein Rap-2b GN=RAP2B, Ig heavy chain V-III region VH26, Serum amyloid A-4 protein GN=SAA4, Alpha-actinin-4 GN=ACTN4, Ig heavy chain V-III region WEA, Guanine nucleotide-binding protein G (k) subunit alpha GN=GNAI3, Ig kappa chain V-III region NG9 (Fragment), HLA class I histocompatibility antigen, Cw-17 alpha chain GN=HLA-C, HLA class I histocompatibility antigen, A-68 alpha chain GN=HLA-A, Fructose-bisphosphate aldolase A GN=ALDOA, Syntenin-1 GN=SDCBP, Beta-actin-like protein 2 GN=ACTBL2, Mucin-1 GN=MUC1, Spondin-1 GN=SPON1, Ig heavy chain V-III region TUR, Cell division control protein 42 homolog GN-CDC42, Apolipoprotein C-III GN=APOC3, Extracellular superoxide dismutase [Cu-Zn] GN═SOD3, Guanine nucleotide-binding protein subunit alpha-11 GN=GNA11, Plasma kallikrein GN=KLKB1, G-protein coupled receptor family C group 5 member C GN=GPRC5C, Antithrombin-III GN=SERPINCI, HLA class I histocompatibility antigen, A-32 alpha chain GN=HLA-A, Ig gamma-4 chain C region GN=IGHG4, Ig kappa chain V-IV region JI, C-reactive protein GN=CRP, Cofilin-1 GN=CFL1, Chloride intracellular channel protein 5 GN=CLIC5, Sulfhydryl oxidase 1 GN=QSOX1, Apolipoprotein A-II GN=APOA2, Ig heavy chain V-II region ARH-77, Complement decay-accelerating factor GN=CD55, 14-3-3 protein beta/alpha GN=YWHAB, Ubiquitin-40S ribosomal protein S27a GN=RPS27A, 2′, 3′-cyclic-nucleotide 3′-phosphodiesterase GN-CNP, Ig heavy chain V-III region GA, Sodium-dependent phosphate transport protein 2B GN=SLC34A2, Profilin-1 GN=PFN1, EH domain-containing protein 4 GN=EHD4, Ig kappa chain V-III region VG (Fragment), Na(+)/H(+) exchange regulatory cofactor NHE-RF1 GN=SLC9A3R1, Serum amyloid P-component GN=APCS, Tenascin-X GN=TNXB, Ig lambda chain V-III region LOI, Triosephosphate isomerase GN=TPI1, Hemopexin GN=HPX, Beta-2-glycoprotein 1 GN=APOH, Intercellular adhesion molecule 1 GN=ICAM1, Interferon-induced transmembrane protein 1 GN=IFITM1, Ficolin-2 GN=FCN2, Ras-related protein Ral-A GN=RALA, Ig kappa chain V-III region VH (Fragment), Transforming protein RhoA GN=RHOA, Coagulation factor XIII A chain GN=F13A1, Leucine-rich repeat neuronal protein 4 GN=LRRN4, Cartilage acidic protein 1 GN-CRTAC1, HLA class II histocompatibility antigen, DR alpha chain GN=HLA-DRA, Serotransferrin GN=TF, CD81 antigen GN=CD81, Choline transporter-like protein 2 GN=SLC44A2, Syntaxin-binding protein 2 GN=STXBP2, HLA class I histocompatibility antigen, A-2 alpha chain GN=HLA-A, Collagen alpha-2 (VI) chain GN═COL6A2, Desmoplakin GN=DSP, Ig kappa chain V-I region HK102 (Fragment) GN=IGKV1-5, Arrestin domain-containing protein 1 GN=ARRDC1, Macrophage receptor MARCO GN=MARCO, Tetraspanin-1 GN=TSPAN1, Rho-related GTP-binding protein RhoC GN=RHOC, Niban-like protein 1 GN=FAM129B, Ig lambda chain V-IV region Hil, Mannosyl-oligosaccharide 1, 2-alpha-mannosidase IA GN=MAN1A1, 14-3-3 protein gamma GN=YWHAG, CD9 antigen GN=CD9, Junction plakoglobin GN=JUP, Vimentin GN=VIM, Prostaglandin F2 receptor negative regulator GN=PTGFRN, Protein-glutamine gamma-glutamyltransferase 2 GN=TGM2, Ras-related protein R-Ras GN=RRAS, Protein S100-A9 GN=S100A9, Ig kappa chain V-I region BAN, Ig heavy chain V-III region JON, Tumor-associated calcium signal transducer 2 GN=TACSTD2, Ig kappa chain V-III region POM, Polymeric immunoglobulin receptor GN=PIGR, Ig kappa chain V-I region Lay, CD82 antigen GN-CD82, Complement factor H-related protein 5 GN=CFHR5, Ig lambda chain V-III region SH, Calcium and integrin-binding protein 1 GN-CIB1, Choline transporter-like protein 4 GN=SLC44A4, Protein Z-dependent protease inhibitor GN=SERPINA10, Apolipoprotein M GN=APOM, Ras-related protein Rab-8A GN=RAB8A, Claudin-1 GN=CLDN1, Pentraxin-related protein PTX3 GN=PTX3, Peroxiredoxin-6 GN=PRDX6, EGF-containing fibulin-like extracellular matrix protein 1 GN=EFEMP1, 14-3-3 protein theta GN=YWHAQ, Ig kappa chain V-II region MIL, Ras-related protein Rab-1A GN=RABIA, Ig kappa chain V-II region GM607 (Fragment), Aldehyde dehydrogenase family 3 member B1 GN=ALDH3B1, Sodium/potassium-transporting ATPase subunit alpha-1 GN=ATP1A1, Glutathione S-transferase P GN=GSTP1, Complement factor B GN=CFB, Coagulation factor IX GN=F9, Ig kappa chain V-III region CLL, Ig lambda chain V-I region WAH, Ig lambda chain V-IV region MOL, Intestinal-type alkaline phosphatase GN=ALPI, Myeloperoxidase GN=MPO, Nidogen-2 GN=NID2, Peroxiredoxin-1 GN=PRDX1, Carboxypeptidase B2 GN-CPB2, Ig lambda chain V-I region NEWM, Vacuolar protein sorting-associated protein 4B GN=VPS4B, Apolipoprotein C-II GN=APOC2, Ig lambda chain V-VI region EB4, Fructose-bisphosphate aldolase C GN=ALDOC, Glutathione peroxidase 3 GN-GPX3, Ig kappa chain V-I region HK101 (Fragment), Thy-1 membrane glycoprotein GN=THY1, Protein lifeguard 3 GN=TMBIM1, Claudin-3 GN=CLDN3, Ig heavy chain V-III region KOL, Ig kappa chain V-II region FR, Carboxypeptidase N catalytic chain GN-CPN1, G-protein coupled receptor family C group 5 member B GN=GPRC5B, Small integral membrane protein 22 GN=SMIM22, Guanine nucleotide-binding protein G (I)/G(S)/G (O) subunit gamma-12 GN-GNG12, Hornerin GN=HRNR, Ig kappa chain V-I region Mev, Protein S100-A10 GN=S100A10, Ig kappa chain V-I region Walker, Ig kappa chain V-III region IARC/BL41, Putative V-set and immunoglobulin domain-containing-like protein IGHV1OR21-1 GN=IGHV1OR21-1, Coagulation factor X GN=F10, Galectin-3 GN=LGALS3, Ras-related protein Rab-11B GN=RAB11B, Filaggrin-2 GN=FLG2, Ig heavy chain V-III region HIL, Cholesteryl ester transfer protein GN-CETP, Thioredoxin GN=TXN, BTB/POZ domain-containing protein KCTD12 GN=KCTD12, Ig heavy chain V-III region LAY, Integrin beta-1 GN=ITGB1, Ig heavy chain V-III region TRO, Ig heavy chain V-I region Mot, Sialic acid-binding Ig-like lectin 16 GN=SIGLEC16, Ig lambda chain V-I region BL2, and Peroxiredoxin-2 GN=PRDX2.

### Protein List 3 (GN=Indicates Gene Name)

Apolipoprotein B-100 GN=APOB, Complement C3 GN=C3, Complement C4-B GN=C4B, Complement C4-A GN=C4A, Fibronectin GN=FN1, Ceruloplasmin GN=CP, Alpha-2-macroglobulin GN=A2M, Complement C5 GN=C5, Fibrinogen beta chain GN=FGB, Serum albumin GN=ALB, Mucin-5B GN=MUC5B, Apolipoprotein (a) GN=LPA, Inter-alpha-trypsin inhibitor heavy chain H1 GN=ITIH1, C4b-binding protein alpha chain GN-C4BPA, Ig mu chain C region GN=IGHM, Inter-alpha-trypsin inhibitor heavy chain H2 GN=ITIH2, Fibrinogen gamma chain GN=FGG, Pregnancy zone protein GN=PZP, Prothrombin GN=F2, Mucin-16 GN=MUC16, Ig mu heavy chain disease protein, von Willebrand factor GN=VWF, Plasma protease C1 inhibitor GN=SERPING1, Sushi, von Willebrand factor type A, EGF and pentraxin domain-containing protein 1 GN=SVEP1, Complement C1r subcomponent GN=CIR, Apolipoprotein A-I GN=APOA1 IgGFc-binding protein GN=FCGBP Complement factor H GN-CFH Haptoglobin GN=HP Phospholipid transfer protein GN=PLTP Ig alpha-1 chain C region GN=IGHA1 Inter-alpha-trypsin inhibitor heavy chain H4 GN-ITIH4 Inter-alpha-trypsin inhibitor heavy chain H3 GN=ITIH3 Complement C1s subcomponent GN=C1S Alpha-1-antitrypsin GN=SERPINA1 Apolipoprotein E GN-APOE Ig alpha-2 chain C region GN=IGHA2 Complement component C9 GN=C9 Vitronectin GN=VTN Clusterin GN=CLU Vitamin K-dependent protein S GN=PROS1 Serum paraoxonase/arylesterase 1 GN=PON1 Fibrinogen alpha chain GN=FGA CD5 antigen-like GN-CD5L Ig kappa chain C region GN-IGKC Aminopeptidase N GN=ANPEP Ig gamma-1 chain C region GN=IGHG1 Proteoglycan 4 GN=PRG4 Plasminogen GN=PLG Transthyretin GN-TTR Tenascin GN=TNC Haptoglobin-related protein GN=HPR Alpha-1-antichymotrypsin GN=SERPINA3 Actin, cytoplasmic 1 GN=ACTB Galectin-3-binding protein GN=LGALS3BP Versican core protein GN=VCAN Fibulin-1 GN=FBLN1 Heparin cofactor 2 GN=SERPIND1 Carboxypeptidase N subunit 2 GN=CPN2, Apolipoprotein A-IV GN=APOA4, Ig gamma-2 chain C region GN=IGHG2, Insulin-like growth factor-binding protein complex acid labile subunit GN=IGFALS, Ig kappa chain V-I region DEE, Complement component C7 GN-C7, Basement membrane-specific heparan sulfate proteoglycan core protein GN═HSPG2, Target of Nesh-SH3 GN=ABI3BP, Laminin subunit beta-1 GN=LAMB1, Complement component C6 GN-C6, Protein AMBP GN=AMBP, Lipopolysaccharide-binding protein GN=LBP, Vitamin D-binding protein GN=GC, Apolipoprotein L1 GN=APOL1, Tenascin-X GN=TNXB, Kininogen-1 GN=KNG1, Angiotensinogen GN-AGT, Serum amyloid A-1 protein GN=SAA1, Sulfhydryl oxidase 1 GN=QSOX1, Ig lambda-2 chain C regions GN=IGLC2, Immunoglobulin J chain GN=IGJ, Gelsolin GN=GSN, Ezrin GN=EZR, Pyruvate kinase PKM GN=PKM, Immunoglobulin lambda-like polypeptide 5 GN=IGLL5, Laminin subunit gamma-1 GN=LAMC1, Lumican GN=LUM, Collagen alpha-3 (VI) chain GN-COL6A3, Complement component C8 alpha chain GN=C8A, Complement component C8 beta chain GN=C8B, Annexin A2 GN=ANXA2, C4b-binding protein beta chain GN=C4BPB, Coagulation factor IX GN=F9, Plasma kallikrein GN-KLKB1 N-acetylmuramoyl-L-alanine amidase GN=PGLYRP2 Ig kappa chain V-IV region Len Glyceraldehyde-3-phosphate dehydrogenase GN-GAPDH Histidine-rich glycoprotein GN=HRG Ig gamma-3 chain C region GN=IGHG3 Ig kappa chain V-III region GOL Protein Z-dependent protease inhibitor GN=SERPINA10 Ig kappa chain V-III region SIE Alpha-2-antiplasmin GN=SERPINF2 Carboxypeptidase N catalytic chain GN-CPN1 Ig kappa chain V-I region OU Ig kappa chain V-I region Scw Ig kappa chain V-I region WEA Hyaluronan-binding protein 2 GN=HABP2 Ig kappa chain V-I region AU EMILIN-2 GN=EMILIN2 Ig kappa chain V-I region Roy Complement component C8 gamma chain GN=C8G Monocyte differentiation antigen CD14 GN=CD14 Antithrombin-III GN=SERPINC1 Apolipoprotein M GN=APOM Apolipoprotein A-II GN=APOA2 Annexin A1 GN=ANXA1 Ig kappa chain V-III region HIC Actin, alpha cardiac muscle 1 GN=ACTC1 Thrombospondin-2 GN=THBS2 Biotinidase GN=BTD Agrin GN=AGRN Carboxypeptidase B2 GN-CPB2 Ig heavy chain V-III region BUT Laminin subunit alpha-4 GN=LAMA4 Mannosyl-oligosaccharide 1, 2-alpha-mannosidase IA GN=MANIA1 Alpha-enolase GN-ENO1, Ig kappa chain V-III region B6, Serotransferrin GN=TF, Alpha-1-acid glycoprotein 2 GN=ORM2, Phosphoglycerate kinase 1 GN=PGK1, Ig kappa chain V-I region Ni, Hemopexin GN=HPX, Coagulation factor V GN=F5, Cartilage acidic protein 1 GN=CRTAC1, Serum amyloid A-4 protein GN=SAA4, Ig gamma-4 chain C region GN=IGHG4, Tubulin beta chain GN=TUBB, Ig heavy chain V-III region VH26, Ig kappa chain V-II region TEW, Prominin-1 GN=PROM1, Ig kappa chain V-I region EU, Tubulin alpha-1B chain GN=TUBA1B, Ig kappa chain V-I region CAR, Prenylcysteine oxidase 1 GN=PCYOX1, C-reactive protein GN=CRP, Myosin-9 GN=MYH9, Thrombospondin-4 GN=THBS4, Ig heavy chain V-III region BRO, Coagulation factor X GN=F10, Mucin-4 GN=MUC4, 5′-nucleotidase GN=NT5E, Complement C1q subcomponent subunit B GN=C1QB, Apolipoprotein D GN=APOD, Heat shock protein HSP 90-beta GN═HSP90AB1, Afamin GN=AFM, Apolipoprotein C-III GN=APOC3, Ig heavy chain V-III region TUR, Ig heavy chain V-III region GAL, Serum amyloid P-component GN=APCS Retinoic acid-induced protein 3 GN-GPRC5A Ig heavy chain V-III region WEA Beta-2-glycoprotein 1 GN=APOH Ig heavy chain V-III region CAM Complement C1q subcomponent subunit C GN=C1QC Alpha-2-HS-glycoprotein GN=AHSG Guanine nucleotide-binding protein G (i) subunit alpha-2 GN-GNAI2 Glutathione peroxidase 3 GN=GPX3 Complement factor B GN-CFB Adipocyte plasma membrane-associated protein GN=APMAP Ig kappa chain V-III region NG9 (Fragment) Cartilage oligomeric matrix protein GN-COMP Ig heavy chain V-I region V35 Phosphatidylinositol-glycan-specific phospholipase D GN-GPLD1 Heat shock protein HSP 90-alpha GN-HSP90AA1 Ig kappa chain V-IV region JI Complement factor H-related protein 1 GN=CFHR1 Cathepsin B GN=CTSB Spondin-1 GN=SPON1 Ig delta chain C region GN=IGHD Pigment epithelium-derived factor GN=SERPINF1 Complement C1r subcomponent-like protein GN=C1RL Ig heavy chain V-II region ARH-77 Pentraxin-related protein PTX3 GN=PTX3 Mannan-binding lectin serine protease 1 GN=MASP1 Extracellular superoxide dismutase [Cu-Zn] GN═SOD3 Ig heavy chain V-I region HG3 Phosphatidylcholine-sterol acyltransferase GN=LCAT Ig kappa chain V-III region VG (Fragment) Moesin GN=MSN Collagen alpha-1 (VI) chain GN═COL6A1 ADP-ribosylation factor 3 GN=ARF3 Soluble scavenger receptor cysteine-rich domain-containing protein SSC5D GN═SSC5D, BPI fold-containing family B member 1 GN=BPIFB1, Ig lambda chain V-III region SH, Heat shock cognate 71 kDa protein GN═HSPA8, Apolipoprotein C-II GN=APOC2, Guanine nucleotide-binding protein G (I)/G(S)/G (T) subunit beta-2 GN=GNB2, Decorin GN=DCN, Ig lambda chain V-IV region Hil, Ig lambda chain V-III region LOI, Extracellular matrix protein 1 GN=ECM1, Peptidyl-prolyl cis-trans isomerase A GN=PPIA, Lactadherin GN=MFGE8, Macrophage receptor MARCO GN=MARCO, Ficolin-2 GN=FCN2, Apolipoprotein C-I GN=APOC1, Selenoprotein P GN=SEPP1, Complement C2 GN=C2, Retinol-binding protein 4 GN-RBP4, Vitamin K-dependent protein Z GN=PROZ, 14-3-3 protein zeta/delta GN=YWHAZ, EGF-containing fibulin-like extracellular matrix protein 1 GN-EFEMP1, Ig kappa chain V-I region BAN, Apolipoprotein C-IV GN=APOC4, Ig heavy chain V-III region GA, Coagulation factor XIII A chain GN=F13A1, Heat shock 70 kDa protein 1-like GN═HSPA1L, Ig kappa chain V-II region MIL, Ig kappa chain V-III region VH (Fragment), Apolipoprotein F GN=APOF, Ig kappa chain V-I region HK102 (Fragment) GN=IGKV1-5, Ig lambda chain V-VI region EB4, Ubiquitin-40S ribosomal protein S27a GN=RPS27A, Ig kappa chain V-I region Wes, Nidogen-2 GN=NID2, Ig kappa chain V-II region RPMI 6410, G-protein coupled receptor family C group 5 member C GN=GPRC5C, Glutathione S-transferase P GN-GSTP1, Ficolin-3 GN=FCN3, Ig lambda chain V-IV region MOL, Ig kappa chain V-I region HK101 (Fragment), Ig kappa chain V-I region Lay, Mannan-binding lectin serine protease 2 GN=MASP2, Ig lambda chain V-I region WAH, Erythrocyte band 7 integral membrane protein GN=STOM, Prostaglandin-H2 D-isomerase GN=PTGDS, Prolow-density lipoprotein receptor-related protein 1 GN=LRP1, Thymidine phosphorylase GN=TYMP, Ig heavy chain V-III region KOL, Ig kappa chain V-III region CLL, Serum paraoxonase/lactonase 3 GN=PON3, Tumor-associated calcium signal transducer 2 GN=TACSTD2, Ig heavy chain V-III region JON, Fructose-bisphosphate aldolase A GN=ALDOA, Ras-related protein Rap-1b-like protein, Polymeric immunoglobulin receptor GN=PIGR, Plexin domain-containing protein 2 GN=PLXDC2, Lymphatic vessel endothelial hyaluronic acid receptor 1 GN-LYVE1, Syntenin-1 GN=SDCBP, Endoplasmin GN═HSP90B1, Ig heavy chain V-III region TRO, Ig heavy chain V-III region HIL, Complement decay-accelerating factor GN=CD55, Profilin-1 GN=PFN1, Cathepsin LI GN=CTSL, Cathelicidin antimicrobial peptide GN=CAMP, Ig heavy chain V-I region Mot, Syndecan-1 GN=SDC1, and Ig kappa chain V-II region FR.

### Protein List 4 (GN=Indicates Gene Name)

Apolipoprotein B-100 GN=APOB, Complement C4-B GN-C4B, Complement C4-A GN=C4A, Alpha-2-macroglobulin GN=A2M, Ig mu chain C region GN=IGHM, Fibronectin GN=FN1, Complement C3 GN=C3, Pregnancy zone protein GN=PZP, Ig mu heavy chain disease protein, Mucin-16 GN=MUC16, Plasma protease C1 inhibitor GN=SERPING1, Ig kappa chain C region GN=IGKC, C4b-binding protein alpha chain GN=C4BPA, Desmoplakin GN-DSP, CD5 antigen-like GN-CD5L, Tenascin GN=TNC, Annexin A2 GN=ANXA2, Serum albumin GN=ALB, Versican core protein GN=VCAN, Ezrin GN=EZR, Ig gamma-1 chain C region GN=IGHG1, Ig kappa chain V-I region DEE, Collagen alpha-3 (VI) chain GN═COL6A3, Immunoglobulin J chain GN=IGJ, Laminin subunit gamma-1 GN=LAMC1, Galectin-3-binding protein GN=LGALS3BP, Mannose-binding protein C GN=MBL2, Immunoglobulin lambda-like polypeptide 5 GN=IGLL5, Ig kappa chain V-III region B6, Haptoglobin GN-HP, Ig lambda-2 chain C regions GN=IGLC2, Basement membrane-specific heparan sulfate proteoglycan core protein GN═HSPG2, Extracellular superoxide dismutase [Cu-Zn] GN═SOD3, Actin, cytoplasmic 1 GN=ACTB, Apolipoprotein A-I GN=APOA1, IgGFc-binding protein GN=FCGBP, Moesin GN=MSN, Ig alpha-1 chain C region GN=IGHA1, Ig kappa chain V-I region WEA, Complement C5 GN=C5, Haptoglobin-related protein GN=HPR, Ig kappa chain V-I region AU, Ig kappa chain V-I region OU, Ig kappa chain V-IV region Len, Collagen alpha-2 (VI) chain GN-COL6A2, Inter-alpha-trypsin inhibitor heavy chain H4 GN=ITIH4, Ig kappa chain V-I region Scw, Vitamin K-dependent protein S GN=PROS1, Ig heavy chain V-III region BUT, Sodium-dependent phosphate transport protein 2B GN=SLC34A2, Annexin A5 GN=ANXA5, Ig kappa chain V-I region EU, Ig kappa chain V-I region Roy, Collagen alpha-1 (VI) chain GN═COL6A1, Laminin subunit beta-1 GN=LAMB1, Mannan-binding lectin serine protease 1 GN=MASP1, Ig alpha-2 chain C region GN=IGHA2, Radixin GN=RDX, Ig kappa chain V-III region SIE, Ig kappa chain V-III region GOL, Complement component C9 GN=C9, Proteoglycan 4 GN=PRG4, Ig kappa chain V-I region Wes, Syntenin-1 GN=SDCBP, Desmoglein-1 GN=DSG1, Ig lambda-7 chain C region GN=IGLC7, Ig kappa chain V-I region Ni, Ig heavy chain V-III region CAM, Sushi domain-containing protein 2 GN=SUSD2, Mucin-5B GN=MUC5B, Ig heavy chain V-III region BRO, Pyruvate kinase PKM GN=PKM, 14-3-3 protein zeta/delta GN=YWHAZ, Ig kappa chain V-II region TEW, Guanine nucleotide-binding protein G (I)/G(S)/G (T) subunit beta-1 GN=GNB1, Annexin A1 GN=ANXA1, Heat shock cognate 71 kDa protein GN═HSPA8, Guanine nucleotide-binding protein G (I)/G(S)/G (T) subunit beta-2 GN=GNB2, Ig gamma-2 chain C region GN=IGHG2, Ig heavy chain V-III region GA, Serum amyloid P-component GN=APCS, Ig heavy chain V-III region TUR, Ubiquitin-40S ribosomal protein S27a GN=RPS27A, Actin, aortic smooth muscle GN=ACTA2, Polymeric immunoglobulin receptor GN=PIGR, Intercellular adhesion molecule 1 GN=ICAM1, Annexin A11 GN=ANXA11, Alpha-1-antichymotrypsin GN=SERPINA3, Inter-alpha-trypsin inhibitor heavy chain H1 GN=ITIH1, Annexin A3 GN=ANXA3, Ig heavy chain V-III region WEA, Agrin GN=AGRN, Unconventional myosin-Ic GN=MYO1C, Ig heavy chain V-I region HG3, ADP-ribosylation factor 3 GN=ARF3, Annexin A4 GN=ANXA4, Triosephosphate isomerase GN=TPI1, Ig kappa chain V-II region MIL, Ig heavy chain V-III region GAL, Lactadherin GN=MFGE8, Mucin-1 GN=MUC1, Ig heavy chain V-II region ARH-77, Ficolin-2 GN=FCN2, Interferon-induced transmembrane protein 3 GN=IFITM3, Alkaline phosphatase, tissue-nonspecific isozyme GN=ALPL, Junction plakoglobin GN=JUP, Guanine nucleotide-binding protein G (i) subunit alpha-2 GN=GNAI2, Choline transporter-like protein 2 GN-SLC44A2, Claudin-1 GN=CLDN1, Sushi, von Willebrand factor type A, EGF and domain-containing protein 1 GN-SVEP1, Apolipoprotein (a) GN=LPA, pentraxin Glyceraldehyde-3-phosphate dehydrogenase GN=GAPDH, Fibrinogen gamma chain GN=FGG, Ig kappa chain V-III region NG9 (Fragment), Ig kappa chain V-IV region STH (Fragment), Claudin-6 GN=CLDN6, G-protein coupled receptor family C group 5 member C GN=GPRC5C, CD9 antigen GN-CD9, Ig kappa chain V-III region POM, HLA class I histocompatibility antigen, Cw-2 alpha chain GN=HLA-C, 5′-nucleotidase GN=NT5E, Inter-alpha-trypsin inhibitor heavy chain H2 GN=ITIH2, Glucose-6-phosphate isomerase GN-GPI, Claudin-3 GN-CLDN3, Ig lambda chain V-III region SH, Ig lambda chain V-III region LOI, Profilin-1 GN=PFN1, Protein AMBP GN=AMBP, Programmed cell death 6-interacting protein GN=PDCD6IP, Erythrocyte band 7 integral membrane protein GN=STOM, Retinoic acid-induced protein 3 GN=GPRC5A, Ig kappa chain V-III region VH (Fragment), 14-3-3 protein epsilon GN=YWHAE, Ras-related protein Rap-1b GN=RAP1B, Apolipoprotein D GN=APOD, Ig heavy chain V-III region VH26, Alpha-enolase GN-ENO1, Ras-related C3 botulinum toxin substrate 1 GN=RAC1, Aminopeptidase N GN=ANPEP, Leukocyte surface antigen CD47 GN=CD47, Alpha-1-antitrypsin GN-SERPINA1, Ig lambda chain V-IV region Hil, Golgi-associated plant pathogenesis-related protein 1 GN=GLIPR2, Protein-glutamine gamma-glutamyltransferase E GN=TGM3, Ig kappa chain V-I region HK102 (Fragment) GN=IGKV1-5, Guanine nucleotide-binding protein G(s) subunit alpha isoforms XLas GN-GNAS, Ig kappa chain V-III region CLL, Lipopolysaccharide-binding protein GN=LBP, Fibulin-1 GN=FBLN1, Protein-glutamine gamma-glutamyltransferase K GN=TGM1, Ig lambda chain V-VI region EB4, Phospholipid scramblase 1 GN=PLSCR1, Complement C1q subcomponent subunit B GN=C1QB, ADP-ribosylation factor 6 GN=ARF6, Ig kappa chain V-III region IARC/BL41, Complement C1q subcomponent subunit C GN=C1QC, Complement component C8 beta chain GN=C8B, Ig lambda chain V-I region WAH, Ig kappa chain V-I region BAN, Prominin-1 GN=PROM1, Heat shock 70 kDa protein 6 GN═HSPA6, von Willebrand factor GN=VWF, Ras-related protein Rab-15 GN=RAB15, Ig kappa chain V-I region Lay, Tumor-associated calcium sign, al transducer 2 GN=TACSTD2 Ig lambda chain V-IV region MOL, Ig lambda chain V-I region NEWM, Ras-related protein Rab-13 GN=RAB13, C-reactive protein GN=CRP, Ig lambda chain V-I region NEW, ATP synthase subunit beta, mitochondrial GN=ATP5B, Complement component C8 gamma chain GN=C8G, Choline transporter-like protein 4 GN=SLC44A4, Tetraspanin-1 GN=TSPAN1, HLA class II histocompatibility antigen, DR alpha chain GN=HLA-DRA, Mannan-binding lectin serine protease 2 GN=MASP2, Ig heavy chain V-III region TRO, Acid sphingomyelinase-like phosphodiesterase 3b GN-SMPDL3B, Thy-1 membrane glycoprotein GN=THY1, Cystatin-A GN-CSTA, Myeloid-associated differentiation marker GN=MYADM, Ig kappa chain V-I region Mev, G-protein coupled receptor family C group 5 member B GN-GPRC5B, Ig heavy chain V-III region KOL, Ig kappa chain V-III region VG (Fragment), Ig kappa chain V-I region HK101 (Fragment), Desmocollin-1 GN=DSC1, Epithelial cell adhesion molecule GN=EPCAM, Thioredoxin GN=TXN, Laminin subunit alpha-2 GN=LAMA2, Peroxiredoxin-1 GN=PRDX1, Trypsin-3 GN=PRSS3, Lipolysis-stimulated lipoprotein receptor GN=LSR, Ig heavy chain V-III region JON, Ig heavy chain V-III region HIL, Transmembrane emp24 domain-containing protein 9 GN=TMED9, Cofilin-1 GN=CFL1, Dermcidin GN=DCD, Peptidyl-prolyl cis-trans isomerase A GN=PPIA, Ig heavy chain V-I region V35, Elongation factor 1-alpha 2 GN=EEF1A2, HLA class II histocompatibility antigen, DRB1-15 beta chain GN=HLA-DRB1, Phospholipid transfer protein GN=PLTP, Ig kappa chain V-I region Ka, Vitronectin GN=VTN, Complement factor H-related protein 5 GN=CFHR5, Guanine nucleotide-binding protein subunit alpha-11 GN=GNA11, Complement decay-accelerating factor GN-CD55, Ig kappa chain V-II region FR, Cellular retinoic acid-binding protein 2 GN-CRABP2, Ig lambda chain V region 4A, Pentraxin-related protein PTX3 GN=PTX3, and Ig lambda chain V-I region BL2.

### Protein List 5 (GN=Indicates Gene Name)

Apolipoprotein B-100 GN=APOB, Complement C3 GN=C3, Complement C4-B GN-C4B, Complement C4-A GN=C4A, Fibronectin GN=FN1, Alpha-2-macroglobulin GN=A2M, Complement C5 GN=C5, Fibrinogen beta chain GN=FGB, Basement membrane-specific heparan sulfate proteoglycan core protein GN═HSPG2, Serum albumin GN=ALB, Pregnancy zone protein GN=PZP, Ceruloplasmin GN-CP, Ig mu chain C region GN=IGHM, Tenascin GN=TNC, C4b-binding protein alpha chain GN=C4BPA, Inter-alpha-trypsin inhibitor heavy chain H1 GN=ITIH1, Plasma protease C1 inhibitor GN=SERPING1, Fibrinogen gamma chain GN=FGG, IgGFc-binding protein GN-FCGBP, Ig mu heavy chain disease protein, Vitamin K-dependent protein S GN=PROS1, von Willebrand factor GN=VWF, Complement factor H GN-CFH, Inter-alpha-trypsin inhibitor heavy chain H4 GN=ITIH4, Complement C1r subcomponent GN=C1R, Ig alpha-1 chain C region GN=IGHA1, Apolipoprotein A-I GN=APOA1, Complement C1s subcomponent GN=C1S, Haptoglobin GN=HP, Galectin-3-binding protein GN=LGALS3BP, Inter-alpha-trypsin inhibitor heavy chain H3 GN=ITIH3, Ig gamma-1 chain C region GN=IGHG1, Alpha-1-antitrypsin GN=SERPINA1, Ig alpha-2 chain C region GN-IGHA2, Ig kappa chain C region GN=IGKC, Agrin GN=AGRN, Complement component C9 GN=C9, Haptoglobin-related protein GN=HPR, Apolipoprotein (a) GN=LPA, Transthyretin GN=TTR, CD5 antigen-like GN-CD5L, Antithrombin-III GN=SERPINC1, Mucin-16 GN=MUC16, Plasminogen GN=PLG, Alpha-1-antichymotrypsin GN=SERPINA3, Inter-alpha-trypsin inhibitor heavy chain H2 GN=ITIH2, Vitamin D-binding protein GN=GC, Extracellular superoxide dismutase [Cu-Zn] GN═SOD3, Versican core protein GN=VCAN, Protein AMBP GN=AMBP, Ig kappa chain V-I region DEE, Tenascin-X GN=TNXB, Laminin subunit beta-1 GN=LAMB1, Kininogen-1 GN=KNG1, Collagen alpha-3 (VI) chain GN═COL6A3, Mannose-binding protein C GN=MBL2, Laminin subunit gamma-1 GN=LAMC1, Laminin subunit alpha-4 GN=LAMA4, Complement component C6 GN=C6, Sushi, von Willebrand factor type A, EGF and pentraxin domain-containing protein 1 GN=SVEP1, Ig lambda-2 chain C regions GN=IGLC2, Immunoglobulin J chain GN=IGJ, Fibulin-1 GN=FBLN1, Immunoglobulin lambda-like polypeptide 5 GN=IGLL5, Collagen alpha-1 (XII) chain GN═COL12A1, Proteoglycan 4 GN=PRG4, Periostin GN=POSTN, Transforming growth factor-beta-induced protein ig-h3 GN=TGFBI, Basal cell adhesion molecule GN=BCAM, Mannan-binding lectin serine protease 1 GN=MASP1, Apolipoprotein M GN=APOM, Clusterin GN-CLU, Complement component C7 GN=C7, Actin, cytoplasmic 1 GN=ACTB, Ig kappa chain V-IV region Len, C4b-binding protein beta chain GN-C4BPB, Angiotensinogen GN=AGT, Ig kappa chain V-I region WEA, Ig gamma-3 chain C region GN=IGHG3, Ig kappa chain V-III region GOL, Ig kappa chain V-I region Scw, Hyaluronan-binding protein 2 GN=HABP2, Ig gamma-2 chain C region GN-IGHG2, Ig kappa chain V-I region Roy, Heparin cofactor 2 GN=SERPIND1, Ig kappa chain V-II region TEW, Fibrinogen alpha chain GN=FGA, Ig kappa chain V-III region SIE, Ig kappa chain V-I region EU, Ig kappa chain V-III region B6, Nidogen-2 GN=NID2, Serum amyloid P-component GN=APCS, Ig heavy chain V-III region BUT, Alpha-2-antiplasmin GN=SERPINF2, Vitronectin GN=VTN, Ig kappa chain V-I region Wes, Ig kappa chain V-III region HIC, Collagen alpha-1 (VI) chain GN═COL6A1, Laminin subunit alpha-2 GN=LAMA2, Histidine-rich glycoprotein GN-HRG, Polymeric immunoglobulin receptor GN=PIGR, Carboxypeptidase N subunit 2 GN=CPN2, Ig lambda-7 chain C region GN=IGLC7, Complement component C8 alpha chain GN-C8A, Beta-2-glycoprotein 1 GN=APOH, Complement C1q subcomponent subunit C GN=C1QC, Ig heavy chain V-III region BRO, Phospholipid transfer protein GN=PLTP, Complement component C8 gamma chain GN-C8G, Ig kappa chain V-I region Ni, Ig heavy chain V-III region CAM, Complement component C8 beta chain GN=C8B, Mannan-binding lectin serine protease 2 GN=MASP2, Ig heavy chain V-III region WEA, Vitamin K-dependent protein Z GN-PROZ, Serum amyloid A-4 protein GN=SAA4, Lipopolysaccharide-binding protein GN=LBP, Apolipoprotein A-II GN=APOA2, Apolipoprotein D GN=APOD, Ig heavy chain V-III region TUR, Ig kappa chain V-II region MIL, Target of Nesh-SH3 GN=ABI3BP, EGF-containing fibulin-like extracellular matrix protein 1 GN=EFEMP1, Ig heavy chain V-III region TIL, Ig kappa chain V-III region NG9 (Fragment), Complement factor H-related protein 4 GN-CFHR4, Ig heavy chain V-II region ARH-77, Ig heavy chain V-I region HG3, Serotransferrin GN=TF, C-reactive protein GN-CRP, Ig heavy chain V-III region WAS, Ig kappa chain V-I region HK102 (Fragment) GN-IGKV1-5, Ig kappa chain V-III region VG (Fragment), Ig kappa chain V-I region BAN, Ig lambda chain V-III region SH, Complement factor H-related protein 5 GN=CFHR5, Thrombospondin-2 GN=THBS2, Ig lambda chain V-III region LOI, Ficolin-2 GN=FCN2, Serum amyloid A-1 protein GN-SAA1, Pentraxin-related protein PTX3 GN=PTX3, Ig lambda chain V-IV region Hil, Ubiquitin-40S ribosomal protein S27a GN=RPS27A, Serum paraoxonase/arylesterase 1 GN=PON1, Ig heavy chain V-III region GAL, Complement C1q subcomponent subunit B GN=C1QB, Prothrombin GN=F2, Alpha-1-acid glycoprotein 2 GN=ORM2, Macrophage colony-stimulating factor 1 receptor GN═CSF1R, Carboxypeptidase N catalytic chain GN-CPN1, Apolipoprotein A-IV GN=APOA4, Interferon-induced transmembrane protein 3 GN=IFITM3, Plasma kallikrein GN=KLKB1, Ig lambda chain V-IV region MOL, Apolipoprotein C-I GN=APOC1, Lymphatic vessel endothelial hyaluronic acid receptor 1 GN=LYVE1, Ig heavy chain V-III region JON, Ig kappa chain V-III region CLL, Ig kappa chain V-III region IARC/BL41, Ig kappa chain V-I region Mev, Adiponectin GN=ADIPOQ, Hepatocyte growth factor activator GN-HGFAC, Ig kappa chain V-IV region STH (Fragment), Nidogen-1 GN=NID1, Mucin-5B GN=MUC5B, Apolipoprotein E GN=APOE, Coagulation factor IX GN=F9, Glypican-1 GN-GPC1, Deleted in malignant brain tumors 1 protein GN=DMBT1, Tetraspanin-1 GN=TSPAN1, Ig heavy chain V-III region GA, Ig heavy chain V-III region HIL, ADP-ribosylation factor 3 GN=ARF3, Ig lambda chain V-I region HA, Dermcidin GN=DCD, Lysozyme C GN=LYZ, and Ig lambda chain V-I region BL2.

### Protein List 6 (GN=Indicates Gene Name)

Apolipoprotein B-100 GN=APOB, Complement C4-B GN=C4B, Complement C4-A GN=C4A, Fibronectin GN=FN1, Complement C3 GN-C3, Inter-alpha-trypsin inhibitor heavy chain H1 GN=ITIH1, Collagen alpha-3 (VI) chain GN═COL6A3, Basement membrane-specific heparan sulfate proteoglycan core protein GN═HSPG2, Ig mu chain C region GN=IGHM, Alpha-2-macroglobulin GN=A2M, Laminin subunit gamma-1 GN=LAMC1, C4b-binding protein alpha chain GN-C4BPA, Serum albumin GN=ALB, Apolipoprotein A-I GN=APOA1, Plasma protease C1 inhibitor GN=SERPING1, Agrin GN=AGRN, Ig mu heavy chain disease protein, Inter-alpha-trypsin inhibitor heavy chain H2 GN=ITIH2, Laminin subunit alpha-5 GN=LAMA5 SV=8, Mucin-16 GN=MUC16, Complement C5 GN=C5, Laminin subunit beta-2 GN=LAMB2, Fibrinogen beta chain GN-FGB, Ig kappa chain C region GN=IGKC, Annexin A2 GN=ANXA2, Laminin subunit beta-1 GN=LAMB1, Complement component C9 GN=C9, Fibrinogen gamma chain GN=FGG, Ig alpha-1 chain C region GN=IGHA1, Versican core protein GN=VCAN, Pentraxin-related protein PTX3 GN=PTX3, Tenascin GN=TNC, Alpha-enolase GN=ENO1, Vitamin K-dependent protein S GN=PROS1, Ezrin GN=EZR, Basal cell adhesion molecule GN=BCAM, Moesin GN=MSN, Alpha-1-antitrypsin GN=SERPINA1, Ig gamma-1 chain C region GN=IGHG1, Inter-alpha-trypsin inhibitor heavy chain H3 GN=ITIH3, Actin, cytoplasmic 1 GN=ACTB, Laminin subunit alpha-4 GN=LAMA4, Protein-glutamine gamma-glutamyltransferase K GN=TGM1, Target of Nesh-SH3 GN=ABI3BP, Ceruloplasmin GN-CP, Ig alpha-2 chain C region GN=IGHA2, Ig kappa chain V-I region DEE, Nidogen-1 GN=NID1, Proteoglycan 4 GN=PRG4, CD5 antigen-like GN=CD5L, Ig lambda-2 chain C regions GN=IGLC2, Ig kappa chain V-I region AG, Desmoplakin GN=DSP, Pregnancy zone protein GN=PZP, Ig kappa chain V-IV region Len, Vitronectin GN=VTN, Apolipoprotein E GN=APOE, Immunoglobulin lambda-like polypeptide 5 GN=IGLL5, Collagen alpha-2 (VI) chain GN═COL6A2, Pyruvate kinase PKM GN=PKM, Lipopolysaccharide-binding protein GN=LBP, Ig kappa chain V-I region WEA, Myeloperoxidase GN=MPO, Complement component C6 GN=C6, Ig gamma-3 chain C region GN=IGHG3, Haptoglobin-related protein GN=HPR, Collagen alpha-1 (VI) chain GN═COL6A1, Clusterin GN-CLU, von Willebrand factor GN=VWF, Ig kappa chain V-III region SIE, Programmed cell death 6-interacting protein GN=PDCD6IP, Ig kappa chain V-I region Roy, Prothrombin GN=F2, Ig gamma-2 chain C region GN=IGHG2, Haptoglobin GN=HP, Complement component C8 beta chain GN=C8B, Ig kappa chain V-I region EU, Glyceraldehyde-3-phosphate dehydrogenase GN=GAPDH, Intercellular adhesion molecule 1 GN=ICAM1, Fibrinogen alpha chain GN=FGA, Immunoglobulin J chain GN=IGJ, Complement component C7 GN=C7, Heparin cofactor 2 GN=SERPIND1, CD82 antigen GN-CD82, Phospholipid transfer protein GN=PLTP, Aminopeptidase N GN=ANPEP, Alpha-1-antichymotrypsin GN-SERPINA3, Ig kappa chain V-III region B6, Annexin A1 GN=ANXA1, Complement C1q subcomponent subunit C GN=C1QC, Ig heavy chain V-III region BRO, Ig heavy chain V-III region BUT, Junction plakoglobin GN-JUP, Transthyretin GN=TTR, Erythrocyte band 7 integral membrane protein GN-STOM, Inter-alpha-trypsin inhibitor heavy chain H4 GN=ITIH4, HLA class I histocompatibility antigen, Cw-6 alpha chain GN=HLA-C, Ig gamma-4 chain C region GN-IGHG4, Heat shock cognate 71 kDa protein GN═HSPA8, Apolipoprotein A-II GN=APOA2, Ig heavy chain V-III region TIL, HLA class I histocompatibility antigen, A-68 alpha chain GN=HLA-A, Mannose-binding protein C GN=MBL2, Serum amyloid A-1 protein GN-SAA1, ADP-ribosylation factor 3 GN=ARF3, Complement component C8 gamma chain GN=C8G, Sodium/potassium-transporting ATPase subunit alpha-1 GN=ATP1A1, Ubiquitin-40S ribosomal protein S27a GN=RPS27A, HLA class I histocompatibility antigen, A-33 alpha chain GN=HLA-A, Guanine nucleotide-binding protein G (i) subunit alpha-2 GN=GNAI2, Ig heavy chain V-III region TUR, Complement factor H GN=CFH, Collagen alpha-1(XV) chain GN═COL15A1, Tumor-associated calcium signal transducer 2 GN=TACSTD2, CD81 antigen GN=CD81, Kininogen-1 GN=KNG1, Claudin-1 GN=CLDN1, Retinoic acid-induced protein 3 GN=GPRC5A, Nidogen-2 GN=NID2, Heat shock protein HSP 90-alpha GN═HSP90AA1, Ig heavy chain V-II region ARH-77, Serum amyloid P-component GN=APCS, G-protein coupled receptor family C group 5 member C GN=GPRC5C, Ig heavy chain V-III region VH26, Ras-related protein Rap-1b GN=RAP1B, Actin, aortic smooth muscle GN=ACTA2, Tubulin alpha-1B chain GN=TUBA1B, Fibulin-1 GN=FBLN1, Ig kappa chain V-III region CLL, Complement C1q subcomponent subunit B GN=C1QB, Elongation factor 1-alpha 1 GN=EEF1A1, EH domain-containing protein 1 GN=EHD1, Ig kappa chain V-II region TEW, CD9 antigen GN=CD9, Peroxiredoxin-1 GN=PRDX1, Annexin A5 GN=ANXA5, Claudin-3 GN=CLDN3, Gamma-enolase GN=ENO2, Complement component C8 alpha chain GN=C8A, Prostaglandin F2 receptor negative regulator GN=PTGFRN, Complement C1s subcomponent GN-C1S, Heat shock 70 kDa protein 1A/1B GN═HSPA1A, Histidine-rich glycoprotein GN=HRG, Tetraspanin-1 GN=TSPAN1, Mucin-4 GN=MUC4, Serpin B12 GN=SERPINB12, Azurocidin GN=AZU1, Apolipoprotein C-I GN=APOC1, 14-3-3 protein zeta/delta GN=YWHAZ, Guanine nucleotide-binding protein G (I)/G(S)/G (T) subunit beta-1 GN=GNB1, Desmoglein-1 GN=DSG1, Dermcidin GN=DCD, L-lactate dehydrogenase A chain GN=LDHA, Serum amyloid A-4 protein GN=SAA4, Sodium-dependent phosphate transport protein 2B GN=SLC34A2, Extracellular superoxide dismutase [Cu-Zn] GN═SOD3, Prolactin-inducible protein GN=PIP, Carboxypeptidase B2 GN=CPB2, Ig lambda chain V-III region SH, Rho-related GTP-binding protein RhoC GN=RHOC, EMILIN-2 GN=EMILIN2, Prominin-2 GN=PROM2, Chloride intracellular channel protein 1 GN=CLIC1, Protein AMBP GN=AMBP, Complement factor H-related protein 1 GN-CFHR1, Ficolin-2 GN=FCN2, Cell division control protein 42 homolog GN=CDC42, Apolipoprotein D GN=APOD, Histone H4 GN=HIST1H4A, Solute carrier family 2, facilitated glucose transporter member 1 GN=SLC2A1, Ig kappa chain V-III region VG (Fragment), Ras-related C3 botulinum toxin substrate 2 GN=RAC2, Ig kappa chain V-III region NG9 (Fragment), G-protein coupled receptor family C group 5 member B GN=GPRC5B, C4b-binding protein beta chain GN=C4BPB, C-reactive protein GN-CRP, Ras-related protein Rab-15 GN=RAB15, Ig heavy chain V-III region KOL, Peptidyl-prolyl cis-trans isomerase A GN=PPIA, Neutral amino acid transporter B (0) GN=SLC1A5, Epithelial cell adhesion molecule GN=EPCAM, Coagulation factor IX GN=F9, Glutathione S-transferase P GN=GSTP1, Apolipoprotein C-II GN=APOC2, Leukocyte surface antigen CD47 GN-CD47, Ig lambda chain V-III region LOI,

### Protein List 7 (GN=Indicates Gene Name)

Apolipoprotein B-100 GN=APOB, Complement C4-B GN=C4B, Complement C4-A GN=C4A, Fibronectin GN=FN1, Complement C3 GN=C3, Complement C5 GN-C5, Alpha-2-macroglobulin GN=A2M, Basement membrane-specific heparan sulfate proteoglycan core protein GN═HSPG2, Fibrinogen beta chain GN=FGB, Inter-alpha-trypsin inhibitor heavy chain H1 GN=ITIH1, Serum albumin GN=ALB, Plasma protease C1 inhibitor GN=SERPING1, C4b-binding protein alpha chain GN-C4BPA, Ig mu chain C region GN=IGHM, Agrin GN=AGRN, Ceruloplasmin GN=CP, Tenascin GN=TNC, Fibrinogen gamma chain GN-FGG, Apolipoprotein A-I GN=APOA1, Inter-alpha-trypsin inhibitor heavy chain H2 GN=ITIH2, Laminin subunit gamma-1 GN=LAMC1, Prothrombin GN=F2, Pregnancy zone protein GN=PZP, Complement component C9 GN-C9, Ig alpha-1 chain C region GN=IGHA1, Laminin subunit beta-1 GN=LAMB1, Vitamin K-dependent protein S GN=PROS1, Alpha-1-antitrypsin GN=SERPINA1, Complement factor H GN=CFH, Ig kappa chain C region GN-IGKC, Haptoglobin GN=HP, Clusterin GN-CLU, Ig gamma-1 chain C region GN-IGHG1, Fibrinogen alpha chain GN=FGA, Transthyretin GN=TTR, Ig alpha-2 chain C region GN-IGHA2, Versican core protein GN=VCAN, Inter-alpha-trypsin inhibitor heavy chain H4 GN=ITIH4, Haptoglobin-related protein GN=HPR, Complement C1s subcomponent GN-CIS, Desmoplakin GN=DSP, Basal cell adhesion molecule GN=BCAM, CD5 antigen-like GN-CD5L, Kininogen-1 GN=KNG1, Complement C1r subcomponent GN-CIR, Laminin subunit alpha-5 GN=LAMA5 SV=8, Vitronectin GN=VTN, Ig lambda-2 chain C regions GN=IGLC2, Phospholipid transfer protein GN-PLTP, Ig gamma-2 chain C region GN=IGHG2, Laminin subunit alpha-4 GN=LAMA4, Serum paraoxonase/arylesterase 1 GN=PON1, Laminin subunit beta-2 GN=LAMB2, Alpha-1-antichymotrypsin GN=SERPINA3, Inter-alpha-trypsin inhibitor heavy chain H3 GN=ITIH3, Pyruvate kinase PKM GN=PKM, Proteoglycan 4 GN=PRG4, Ig gamma-3 chain C region GN=IGHG3, Immunoglobulin lambda-like polypeptide 5 GN=IGLL5, Pentraxin-related protein PTX3 GN=PTX3, C4b-binding protein beta chain GN-C4BPB, Ig kappa chain V-I region AG, Ig kappa chain V-I region WEA, Ig kappa chain V-IV region Len, Antithrombin-III GN=SERPINC1, Immunoglobulin J chain GN=IGJ, Mannose-binding protein C GN=MBL2, Angiotensinogen GN=AGT, Histidine-rich glycoprotein GN=HRG, Ig kappa chain V-I region Roy, Desmoglein-1 GN-DSG1, Apolipoprotein E GN=APOE, Complement component C8 beta chain GN=C8B, Lumican GN=LUM, von Willebrand factor GN=VWF, Ig gamma-4 chain C region GN=IGHG4, Plasminogen GN=PLG, Heat shock protein HSP 90-beta GN═HSP90AB1, Ig kappa chain V-III region B6, Sushi, von Willebrand factor type A, EGF and pentraxin domain-containing protein 1 GN=SVEP1, Apolipoprotein A-II GN=APOA2, Heat shock protein HSP 90-alpha GN═HSP90AA1, Plasma kallikrein GN=KLKB1, Actin, cytoplasmic 1 GN=ACTB, Ig kappa chain V-III region SIE, Fibulin-1 GN=FBLN1, Serum amyloid P-component GN=APCS, Serum amyloid A-1 protein GN=SAA1, Ig kappa chain V-I region EU, IgGFc-binding protein GN=FCGBP, Nidogen-1 GN=NID1, C-reactive protein GN-CRP, Complement component C8 gamma chain GN=C8G, Complement component C6 GN-C6, Ig heavy chain V-III region BUT, Complement component C7 GN-C7, Heparin cofactor 2 GN=SERPIND1, Target of Nesh-SH3 GN=ABI3BP, Protein AMBP GN=AMBP, Mucin-16 GN=MUC16, Serum amyloid A-4 protein GN=SAA4, Retinoic acid receptor responder protein 1 GN=RARRES1, Ig heavy chain V-III region BRO, Junction plakoglobin GN=JUP, Extracellular superoxide dismutase [Cu-Zn] GN═SOD3, Ig heavy chain V-III region VH26, Ig heavy chain V-III region TIL, Apolipoprotein D GN=APOD, Vitamin D-binding protein GN-GC, Apolipoprotein M GN=APOM, Ig heavy chain V-III region TUR, Carboxypeptidase N subunit 2 GN=CPN2, Complement C1q subcomponent subunit C GN=C1QC, Galectin-3-binding protein GN=LGALS3BP, Hemopexin GN=HPX, Ig kappa chain V-III region NG9 (Fragment), Spondin-1 GN=SPON1, Collagen alpha-1(XV) chain GN═COL15A1, Myeloperoxidase GN-MPO, Complement C1q subcomponent subunit B GN=C1QB, Ig lambda chain V-III region LOI, Ig kappa chain V-II region TEW, Carboxypeptidase B2 GN-CPB2, Coagulation factor IX GN=F9, Complement component C8 alpha chain GN-C8A, Ig heavy chain V-II region ARH-77, Dermcidin GN=DCD, Ig heavy chain V-III region GAL, Apolipoprotein A-IV GN=APOA4, Serotransferrin GN=TF, Serpin B12 GN=SERPINB12, Ig kappa chain V-III region CLL, Apolipoprotein C-I GN=APOC1, Lipopolysaccharide-binding protein GN=LBP, Serum amyloid A-2 protein GN=SAA2, Nidogen-2 GN=NID2, Glyceraldehyde-3-phosphate dehydrogenase GN-GAPDH, Complement factor H-related protein 1 GN-CFHR1, Ig delta chain C region GN=IGHD, Ig kappa chain V-III region VG (Fragment), Alpha-2-antiplasmin GN=SERPINF2, Ig lambda chain V-IV region Hil, Collagen alpha-1(XVIII) chain GN-COL18A1, Trypsin-3 GN=PRSS3, Glypican-1 GN=GPC1, Prolactin-inducible protein GN=PIP, Complement component C1q receptor GN=CD93, Ig lambda chain V-I region HA, Deleted in malignant brain tumors 1 protein GN=DMBT1, and Glutathione S-transferase P GN=GSTP1.

1. A method comprising:
subjecting a biological sample comprising a target protein to size-exclusion chromatography;
obtaining from the size-exclusion chromatography a size-exclusion chromatography sample comprising the target protein;
preparing from the size-exclusion chromatography sample a mass-spectrometry sample comprising the target protein or a fragment thereof; and
detecting the target protein or the fragment thereof in the mass-spectrometry sample using mass spectrometry.

2. The method of claim 1, further comprising quantitating the target protein or the fragment thereof in the mass-spectrometry sample using mass spectrometry.

3. The method of claim 1, wherein the size-exclusion chromatography is employed with a resin having a globular protein permeation limit less than 250 kDa.

4. The method of claim 1, wherein the size-exclusion chromatography is employed with a resin having a globular protein exclusion limit of at least 2,500 kDa and up to 40,000 kDa.

5. (canceled)

6. The method of claim 1, wherein the size-exclusion chromatography is performed with an elution buffer having a molar ionic strength from 0.13 to 0.17 M.

7. The method of claim 1, wherein the biological sample is not subjected to ion exchange chromatography prior to the size-exclusion chromatography.

8. The method of claim 1, wherein the biological sample is not subjected to protein adsorption depletion prior to the size-exclusion chromatography.

9. The method of claim 1, wherein the size-exclusion chromatography sample, the mass-spectrometry sample, and any processed forms thereof are not subjected to protein adsorption depletion prior to the detecting.

10. The method of claim 1, wherein the preparing the mass-spectrometry sample comprises subjecting the size-exclusion chromatography sample to protein adsorption depletion, wherein the protein adsorption depletion specifically targets any 1 or more, any 2 or more, any 3 or more, any 4 or more, any 5 or more, any 6 or more, any 7 or more, any 8 or more, any 9 or more, any 10 or more, any 11 or more, any 12 or more, any 13 or more, or each of albumin, IgA, IgD, IgE, IgG, IgG free light chains, IgM, alpha-1-acid glycoprotein, alpha-1-antitrypsin, alpha-2-macroglobulin, apolipoprotein A1, fibrinogen, haptoglobin, and transferrin.

11. The method of claim 1, wherein the biological sample is a direct biological fluid sample or an indirect biological sample prepared from a direct biological fluid sample.

12. The method of claim 1, wherein the biological sample is selected from a blood sample and an ascites fluid sample.

13-15. (canceled)

16. The method of claim 1, wherein an amount of the biological sample subjected to the size-exclusion chromatography corresponds to a volume of a direct biological fluid sample no greater than 5 mL.

17. (canceled)

18. The method of claim 1, wherein the target protein is a mucin.

19. The method of claim 1, wherein the target protein is MUC16.

20. The method of claim 19, wherein the size-exclusion chromatography sample has CA125 purity at least 20-fold higher than CA125 purity of the biological sample.

21. The method of claim 19, wherein the method is capable of enriching CA125 purity in the size-exclusion chromatography sample with respect to the biological sample at CA125 concentration ranges in the biological sample ranging from 30 U/mL to 30,000 U/mL.

22. The method of claim 19, wherein the method is capable of detecting MUC16 at CA125 concentration ranges in the biological sample ranging from 30 U/mL to 30,000 U/mL.

23. The method of claim 19, further comprising quantitating the MUC16 in the mass-spectrometry sample using mass spectrometry, wherein the quantitation of MUC16 with the mass spectrometry correlates with immunoassay CA125 counts in the biological sample at CA125 concentration ranges in the biological sample ranging from 30 U/mL to 30,000 U/mL.

24. The method of claim 1, wherein the size-exclusion chromatography is performed with a disposable size-exclusion chromatography column fed by gravity flow and having a length less than 80 cm.

25. (canceled)

26. The method of claim 1, wherein the size-exclusion chromatography sample comprises a void peak sample.
